(19) United States
(12) Patent Application Publication
(10) Pub. No.: US 2016/0032273 A1
(43) Pub. Date: Feb. 4, 2016

(54) CHARACTERIZATION OF MRNA MOLECULES

(71) Applicant: MODERNA THERAPEUTICS, INC., Cambridge, MA (US)

(72) Inventors: Zahra SHAHROKH, Weston, MA (US); Ingo RÖHL, Cambridge, MA (US); Vlad Boris SPIVAK, Medford, MA (US); Tirtha CHAKRABORTY, Cambridge, MA (US); John Grant AUNINS, Cambridge, MA (US)

(21) Appl. No.: 14/777,059

(22) PCT Filed: Mar. 14, 2014
(86) PCT No.: PCT/US14/28276

§ 371 Date: Sep. 15, 2015

Related U.S. Application Data
(60) Provisional application No. 61/798,945, filed on Mar. 15, 2013.

(51) Int. Cl. C12N 15/10; G01N 27/447

(52) U.S. Cl. CPC C12N 15/10; G01N 27/44717

(57) ABSTRACT

The present invention describes methods for the characterization of mRNA molecules during mRNA production. Characterizing mRNA includes processes such as oligonucleotide mapping, reverse transcriptase sequencing, charge distribution analysis, and detection of RNA impurities. Oligonucleotide mapping includes using an RNase to digest antisense duplexes from an RNA transcript, and then subjecting the digested RNA to reverse phase HPLC, anion exchange HPLC, and/or mass spectrometry analysis. Reverse transcriptase sequencing involves reverse transcription of an RNA transcript followed by DNA sequencing. Charge distribution analysis can comprise procedures such as anion exchange HPLC, or capillary electrophoresis. Detection of impurities includes detecting short mRNA transcripts, RNA-RNA hybrids, and RNA-DNA hybrids.

### BACKGROUND OF THE INVENTION

1. Field of the Invention

The invention relates to methods for the characterization of mRNA molecules during the mRNA production process.

2. Description of the Related Art

Confirmation of structural variants of large mRNA such as sequence aborts, heterogeneous polyA tail, or folded structures is necessary for characterization of manufactured mRNA-based products for preclinical and clinical studies to ensure consistency, safety, and activity of the preparations. The large size and structural variants impose a challenge for many of the available analytical tools that do not have the required resolution or sensitivity.

### SUMMARY OF THE INVENTION

The present invention includes methods for characterizing an RNA transcript. In one embodiment, the RNA transcript is between 100 and 10,000 nucleotides in length. In other embodiments, the RNA transcript is between 600 and 10,000, or between 700 and 3,000 nucleotides in length. In another embodiment, the RNA transcript is a full length RNA transcript. In another embodiment, the RNA transcript includes chemically modified ribonucleotides. In an embodiment, the RNA transcript is the product of in vitro transcription using a non-amplified DNA template. In a separate embodiment, an RNA transcript is characterized by obtaining the RNA transcript and characterizing it by determining the RNA transcript sequence, determining the purity of the RNA transcript, or determining the charge heterogeneity of the RNA transcript. These methods can be accomplished by using procedures such as oligonucleotide mapping, reverse transcriptase sequencing, charge distribution analysis, or detection of RNA impurities.

The RNA transcript can be characterized via oligonucleotide mapping in one embodiment. The RNA transcript is contacted with a plurality of nucleotide probes under conditions sufficient to allow hybridization of the probes to the RNA transcript to form duplexes, where each of the nucleotide probes includes a sequence complementary to a different region of the RNA transcript. In some embodiments, the probes are less than 20 nucleotides in length. In further embodiments, the nucleotide probes include at least 8 deoxynucleotides. In further embodiments, at least one of the nucleotide probes comprises a region that is complementary to a region adjacent to the poly-A tail of the RNA transcript. In still further embodiments, the nucleotide probes are complementary to regions no more than 50 nucleotides apart along the RNA transcript. The duplexes are then contacted with an RNase (such as RNase H or RNase T1) under conditions sufficient to allow RNase digestion of the duplexes to form reaction products. Next, the reaction products are analyzed using a procedure such as reverse phase high performance liquid chromatography (RP-HPLC), anion exchange HPLC (AEX), or RP-HPLC coupled to mass spectrometry (MS). Finally, the RNA transcript is characterized by using the analysis of the reaction products to determine the sequence of the RNA transcript.

The RNA transcript can be characterized by reverse transcriptase sequencing in one embodiment. The RNA transcript is contacted with a reverse transcriptase, a set of primers, and deoxyribonucleotides to obtain one or more cDNA samples. The cDNA samples are contacted with a second set of primers under conditions sufficient to allow PCR to occur, where the cDNA sample serves as a template for obtaining a product comprising amplified cDNA. The product is then characterized by analysis using a sequencing procedure such as Sanger sequencing or bidirectional sequencing. In one embodiment, the primers are complementary to the untranslated regions of mRNA. In other embodiments, the primers have sequences comprising either CGTCGAGCTGCAACGTG, CGTCCTGTCCGTCGCAG, TTTTTTTCTTCCTACTCAGGC, and/or GAAATATAAGAGCCACCATGG.

The RNA transcript can be characterized by anion exchange HPLC (AEX) in one embodiment. A sample comprising the RNA transcript is contacted with an ion exchange sorbent comprising a positively-charged functional group linked to solid phase media, and the sample is delivered with at least one mobile phase, where the RNA transcript in the sample binds the positively-charged functional group of the ion exchange sorbent. In one embodiment, the sample is delivered under denaturing conditions, for example, the sample can be contacted with urea. In other embodiments, the mobile phase is a Tris-EDTA-acetonitrile buffered mobile phase, or there are two mobile phases made of Tris-EDTA-acetonitrile. In other embodiments, the mobile phase comprises a chaotropic salt, such as sodium perchlorate. The ion exchange sorbent elutes a portion of the sample comprising the RNA transcript and one or more separate portions of the sample comprising any impurities. At least one aspect of the portion of the sample comprising the RNA transcript and the separate portions of the sample comprising the impurities are then analyzed, where the aspect is charge heterogeneity of the RNA transcript, mass heterogeneity of the RNA transcript, process intermediates, impurities, or degradation products. The RNA transcript is then characterized by using the analysis to determine the charge heterogeneity of the RNA transcript.

The RNA transcript can be characterized by capillary gel electrophoresis in certain embodiments. A sample comprising the RNA transcript is delivered into a capillary with an electrolyte medium. In one embodiment, the sample is delivered under denaturing conditions. An electric field is applied to the capillary that causes the RNA transcript to migrate through the capillary, where the RNA transcript has a different electrophoretic mobility than any impurities such that the RNA transcript migrates through the capillary at a rate that is different from a rate at which the impurities migrate through the capillary. In one embodiment, the electrophoretic mobility of the RNA transcript is proportional to a mass and an ionic charge of the RNA transcript and inversely proportional to frictional forces in the electrolyte medium. Then, a portion of the sample comprising the RNA transcript and one or more separate portions of the sample comprising the impurities are collected from the capillary. An aspect (such as charge heterogeneity) of the sample comprising the RNA transcript and the portion of the sample comprising the impurities is analyzed. The RNA transcript is then characterized by using the analysis to determine the charge distribution of the RNA transcript and the impurities.

The RNA transcript can be characterized by detection of RNA impurities, including detecting short mRNA transcripts, detecting RNA-RNA and RNA-DNA hybrids, and detecting aberrant nucleotides. In one embodiment, detecting short mRNA transcripts includes denaturing the RNA transcript, and subjecting the denatured RNA transcript to HPLC analysis, where the HPLC analysis quantifies any short mRNA transcript impurities. In an embodiment, the HPLC analysis is reverse phase HPLC. In another embodiment, the reverse phase HPLC analysis is followed by tandem mass spectrometry, where the tandem mass spectrometry identifies any impurities.

In another embodiment, detecting RNA-RNA and RNA-DNA hybrids comprises subjecting the RNA transcript to treatment with urea and EDTA, subjecting the treated RNA transcript to spin filtration, where the filtrate retains a product comprising the impurities, analyzing the product using HPLC, and using the analysis of the product to determine the purity of the RNA transcript, whereby the analysis comprises identification of any RNA-RNA and RNA-DNA hybrids in the product. In some embodiments, the HPLC analysis includes procedures such as anion exchange-HPLC, ion pair reverse phase-HPLC, and electrospray ionization mass spectrometry.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 illustrates a schematic of a primary nucleotide construct, in accordance with an embodiment of the invention.

FIG. 2 illustrates a sequence identity comparison of a GCSF linearized plasmid, in accordance with an embodiment of the invention.

FIG. 3 illustrates a sequence identity comparison of a GCSF PCR product, in accordance with an embodiment of the invention.

FIG. 4 illustrates template sequences and an alignment map of primers for mRNA sequencing, in accordance with an embodiment of the invention.

FIG. 5 illustrates total contiguous sequencing coverage of an mRNA template, in accordance with an embodiment of the invention.

FIG. 6 illustrates mRNA sequence coverage, where the highlighted nucleotides are those that have been sequenced and constitute 91% of the total sequence excluding the polyA tail, and where 100% identity has been established at 100% coverage for the protein coding region, in accordance with an embodiment of the invention.

FIG. 7 illustrates an anion exchange HPLC profile, where an 899 nucleotide mRNA hybridized with 18 nucleotide antisense molecules (RNA or DNA) has been treated with RNase H, in accordance with an embodiment of the invention.

FIG. 8 illustrates a capillary gel electrophoresis profile of tail-less Factor IX mRNA under denaturing conditions, in accordance with an embodiment of the invention.

FIG. 9 illustrates a capillary gel electrophoresis profile of a poly-A tail containing Factor IX mRNA and tail-less Factor IX mRNA under denaturing conditions, in accordance with an embodiment of the invention.

FIG. 10 illustrates a standard co-injection strategy for differentiating mRNA species, in accordance with an embodiment of the invention.

FIG. 11 illustrates a capillary gel electrophoresis profile showing the resolution of two mRNAs, tail-less GCSF and tail-less Factor IX, in accordance with an embodiment of the invention.

FIG. 12 illustrates the reproducibility of relative migration time of 9 repeat injections of ssRNA ladder ranging from 100 to 1000 nucleotides together with Factor IX mRNA. There was a bout a 0.2% relative standard deviation for the relative migration time of the mRNA using ssRNA with n >300 nt and 1.2% for n<300 nt as reference, in accordance with an embodiment of the invention.

### DETAILED DESCRIPTION OF THE INVENTION

Briefly, and as described in more detail below, described herein are methods for characterizing large mRNA transcripts using procedures such as oligonucleotide mapping, reverse transcriptase sequencing, charge distribution analysis, and detection of RNA impurities. Analyses of these procedures are performed using a variety of techniques, including high performance liquid chromatography (HPLC), anion exchange HPLC, capillary electrophoresis (CE), Sanger sequencing, ion pair reverse phase HPLC, mass spectrometry, and electrospray ionization mass spectrometry.

### DEFINITIONS

Terms used in the claims and specification are defined as set forth below unless otherwise specified.

At various places in the present specification, substituents of compounds of the present disclosure are disclosed in groups or in ranges. It is specifically intended that the present disclosure include each and every individual subcombination of the members of such groups and ranges. For example, the term “C1-6 alkyl” is specifically intended to individually disclose methyl, ethyl, C3 alkyl, C4 alkyl, C5 alkyl, and C6 alkyl.

About: As used herein, the term “about” means+/−10% of the recited value.

Approximately: As used herein, the term “approximately” or “about,” as applied to one or more values of interest, refers to a value that is similar to a stated reference value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” “coupled,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It can also suggest ionic or hydrogen bonding or a hybridization based connectivity sufficiently stable such that the “associated” entities remain physically associated.

Amino: the term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkcycloalkyl, carboxyalkyl, sulfoalkyl, heterocyclyl (e.g., heteroaryl), or alkheterocyclyl (e.g., alkheteroaryl), wherein each of these recited R^{N1} groups can be optionally substituted, as defined herein for each group; or two R^{N1} combine to form a heterocyclyl or an N-protecting group, and wherein each R^{N2} is, independently, H, alkyl, or aryl. The amino groups of the invention can be an unsubstituted amino (i.e., —NH₂) or a substituted amino (i.e., —N(R^{N1})₂). In a preferred embodiment, amino is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, carboxyalkyl, sulfoalkyl, or aryl, and each R^{N2} can be H, C₁₋₂₀ alkyl (e.g., C₁₋₆ alkyl), or C₆₋₁₀ aryl.

Label: As used herein, “label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels can be located at any position in the peptides or proteins disclosed herein. They can be within the amino acids, the peptides, or proteins, or located at the N- or C-termini.

DNA template: As used herein, a DNA template refers to a polynucleotide template for RNA polymerase. Typically a DNA template includes the sequence for a gene of interest operably linked to a RNA polymerase promoter sequence.

Digest: As used herein, the term “digest” means to break apart into smaller pieces or components. When referring to polypeptides or proteins, digestion results in the production of peptides. When referring to mRNA, digestion results in the production of oligonucleotide fragments.

Engineered: As used herein, embodiments of the invention are “engineered” when they are designed to have a feature or property, whether structural or chemical, that varies from a starting point, wild type or native molecule.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Fragment: A “fragment,” as used herein, refers to a portion. For example, fragments of proteins can comprise polypeptides obtained by digesting full-length protein isolated from cultured cells.

Gene of interest: As used herein, “gene of interest” refers to a polynucleotide which encodes a polypeptide or protein of interest. Depending on the context, the gene of interest refers to a deoxyribonucleic acid, e.g., a gene of interest in a DNA template which can be transcribed to an RNA transcript, or a ribonucleic acid, e.g., a gene of interest in an RNA transcript which can be translated to produce the encoded polypeptide of interest in vitro, in vivo, in situ or ex vivo. As described in more detail below, a polypeptide of interest includes but is not limited to, biologics, antibodies, vaccines, therapeutic proteins or peptides, etc.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances can have varying levels of purity in reference to the substances from which they have been associated. Isolated substances and/or entities can be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” it is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Modified: As used herein “modified” refers to a changed state or structure of a molecule of the invention. Molecules can be modified in many ways including chemically, structurally, and functionally. In one embodiment, the mRNA molecules of the present invention are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like. For example, a gene of interest operably linked to an RNA polymerase promoter allows transcription of the gene of interest.

Peptide: As used herein, “peptide” is less than or equal to 50 amino acids long, e.g., about 5, 10, 15, 20, 25, 30, 35, 40, 45, or 50 amino acids long.

Poly A tail: As used herein, “poly A tail” refers to a chain of adenine nucleotides. The term can refer to poly A tail that is to be added to an RNA transcript, or can refer to the poly A tail that already exists at the 3′ end of an RNA transcript. As described in more detail below, a poly A tail is typically 5-300 nucleotides in length.

Purified: As used herein, “purify,” “purified,” “purification” means to make substantially pure or clear from unwanted components, material defilement, admixture or imperfection.

RNA transcript: As used herein, an “RNA transcript” refers to a ribonucleic acid produced by an in vitro transcription reaction using a DNA template and an RNA polymerase. As described in more detail below, an RNA transcript typically includes the coding sequence for a gene of interest and a poly A tail. RNA transcript includes an mRNA. The RNA transcript can include modifications, e.g., modified nucleotides. As used herein, the term RNA transcript includes and is interchangeable with mRNA, modified mRNA “mmRNA” or modified mRNA, and primary construct.

Signal Sequences: As used herein, the phrase “signal sequences” refers to a sequence which can direct the transport or localization of a protein.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Stable: As used herein “stable” refers to a compound that is sufficiently robust to survive isolation to a useful degree of purity from a reaction mixture, and preferably capable of formulation into an efficacious therapeutic agent.

Subject: As used herein, the term “subject” or “patient” refers to any organism to which a composition in accordance with the invention can be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Synthetic: The term “synthetic” means produced, prepared, and/or manufactured by the hand of man. Synthesis of polynucleotides or polypeptides or other molecules of the present invention can be chemical or enzymatic.

Transcription factor: As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors can regulate transcription of a target gene alone or in a complex with other molecules.

Unmodified: As used herein, “unmodified” refers to any substance, compound or molecule prior to being changed in any way. Unmodified can, but does not always, refer to the wild type or native form of a biomolecule. Molecules can undergo a series of modifications whereby each modified molecule can serve as the “unmodified” starting molecule for a subsequent modification.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” can mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

### Compositions of the Invention

The present invention provides nucleic acid molecules, specifically polynucleotides, primary constructs and/or mRNA which encode one or more polypeptides of interest. The term “nucleic acid,” in its broadest sense, includes any compound and/or substance that comprise a polymer of nucleotides. These polymers are often referred to as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

In preferred embodiments, the nucleic acid molecule is a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo.

Traditionally, the basic components of an mRNA molecule include at least a coding region, a 5′ UTR, a 3′ UTR, a 5′ cap and a poly-A tail. Building on this wild type modular structure, the present invention expands the scope of functionality of traditional mRNA molecules by providing polynucleotides or primary RNA constructs which maintain a modular organization, but which comprise one or more structural and/or chemical modifications or alterations which impart useful properties to the polynucleotide including, in some embodiments, the lack of a substantial induction of the innate immune response of a cell into which the polynucleotide is introduced. As such, modified mRNA molecules of the present invention are termed “mmRNA.” As used herein, a “structural” feature or modification is one in which two or more linked nucleotides are inserted, deleted, duplicated, inverted or randomized in a polynucleotide, primary construct or mmRNA without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” can be chemically modified to “AT-5meC-G”. The same polynucleotide can be structurally modified from “ATCG” to “ATCCCG”. Here, the dinucleotide “CC” has been inserted, resulting in a structural modification to the polynucleotide.

mRNA Architecture

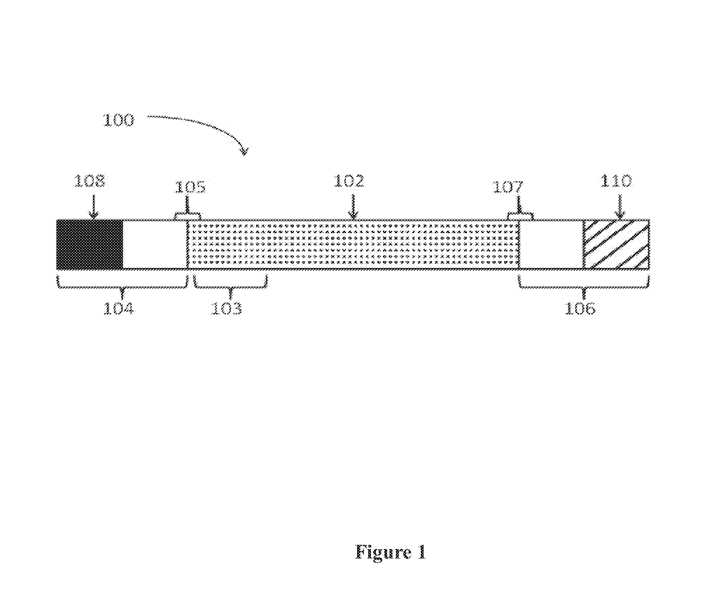

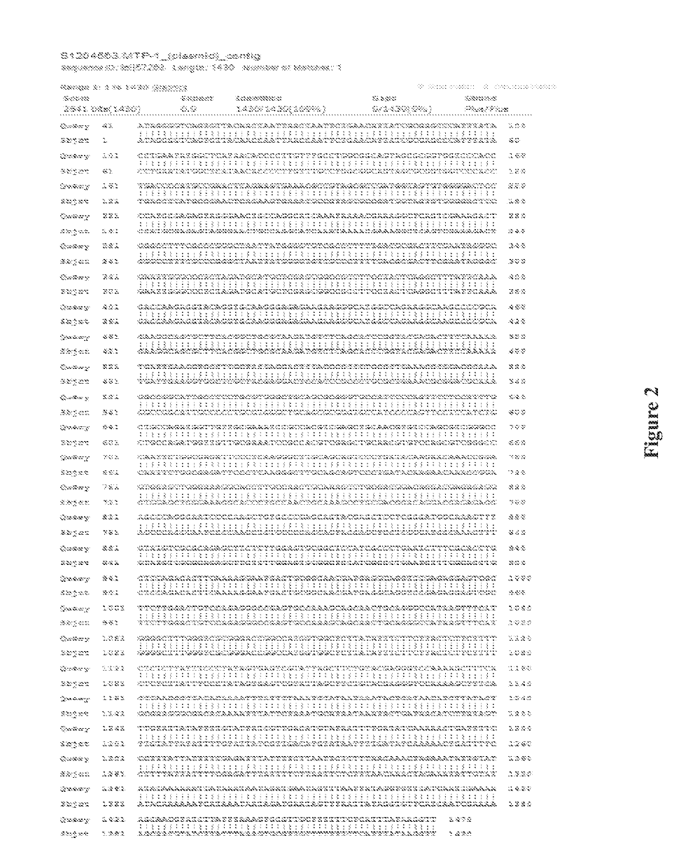

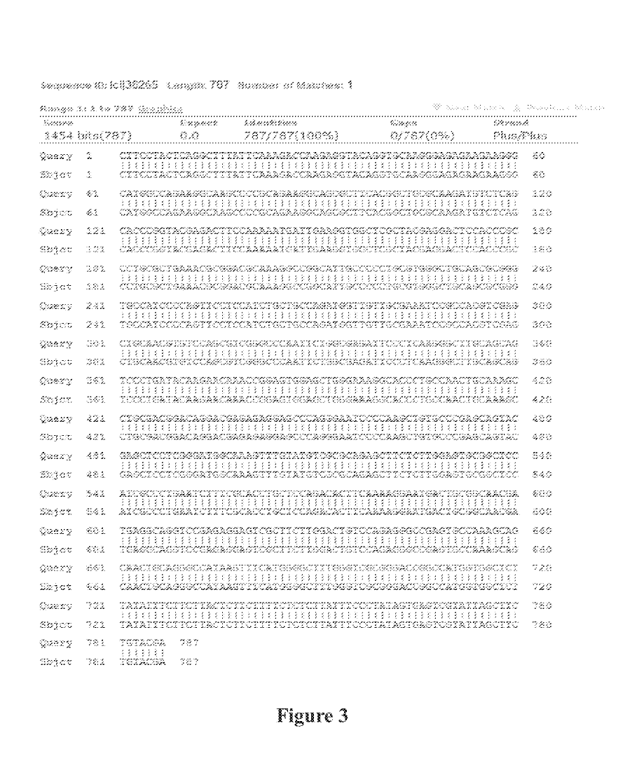

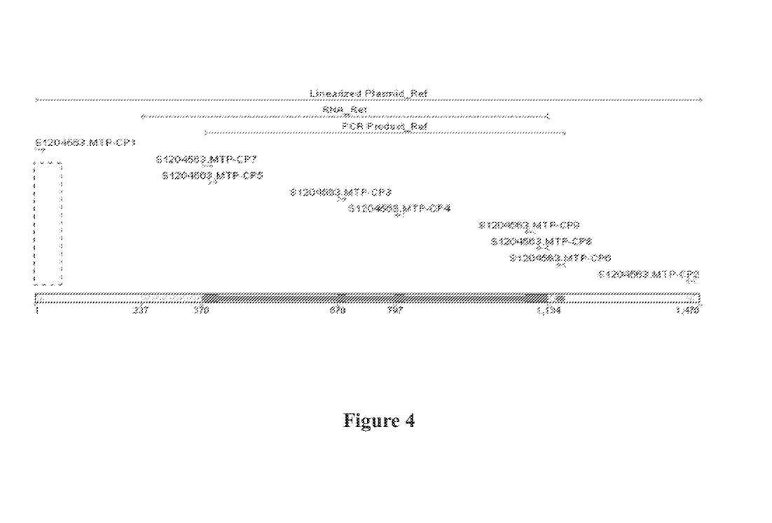

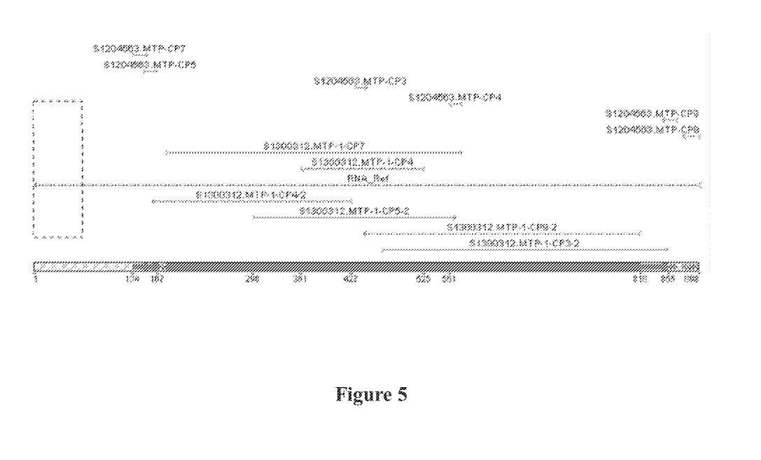

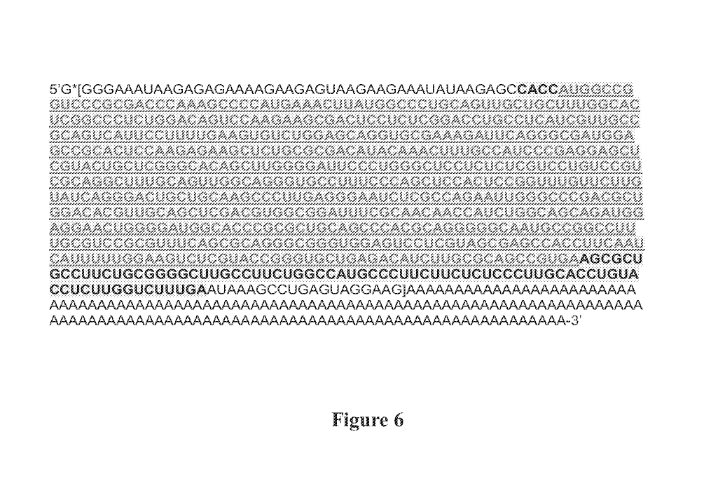

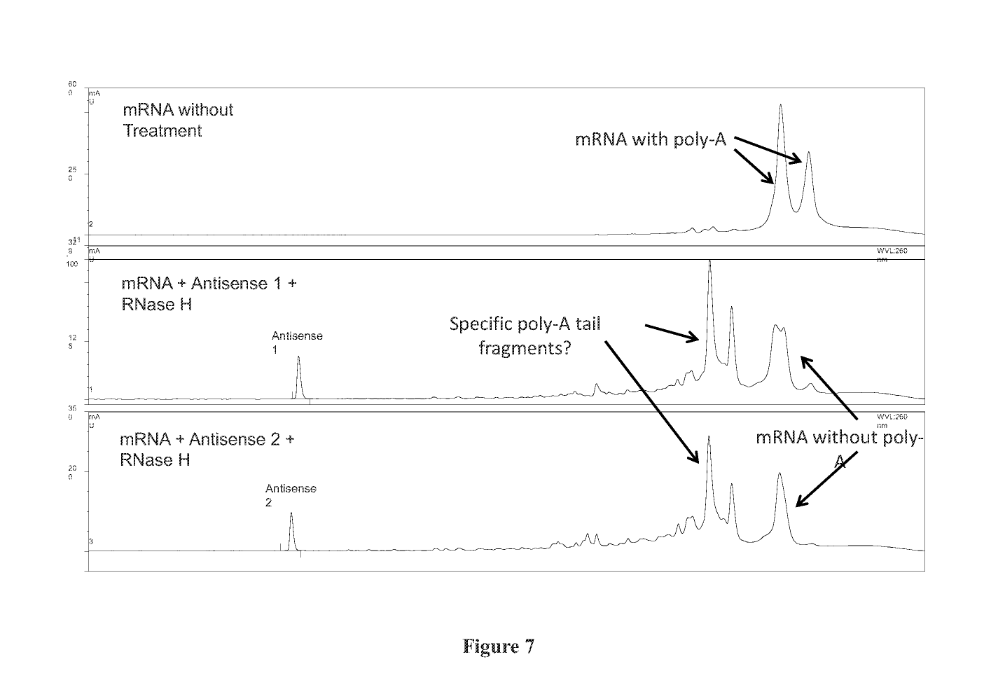

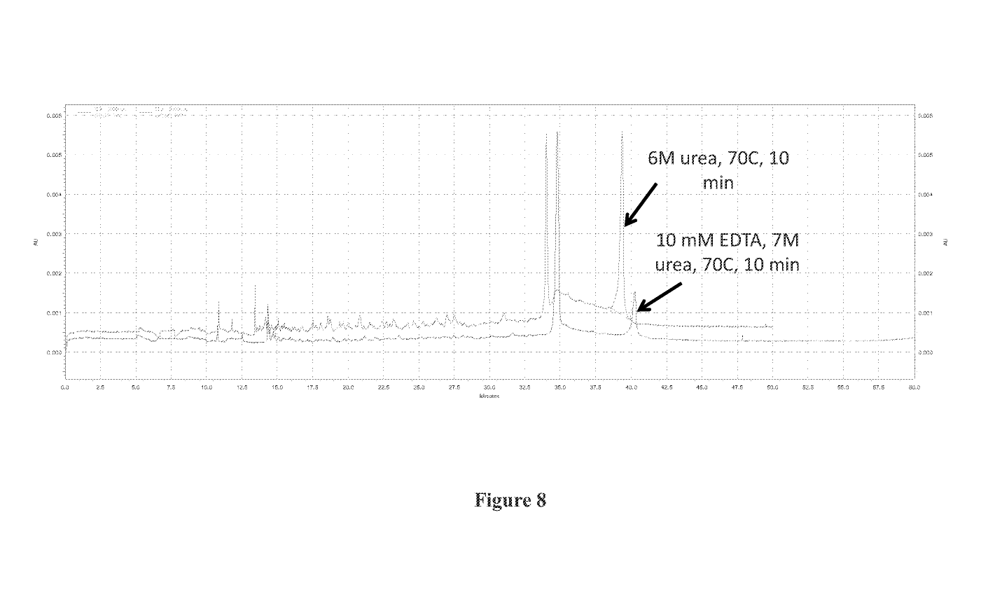

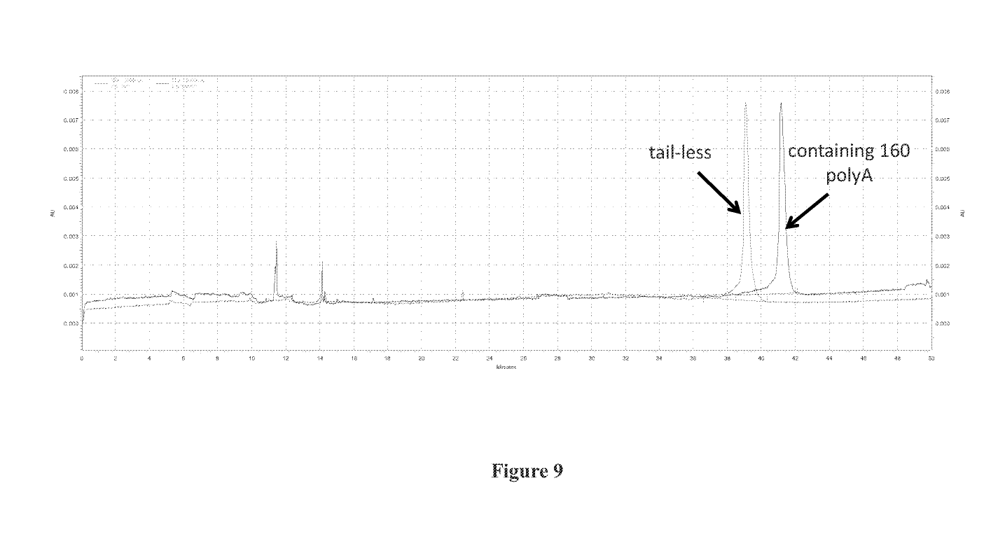

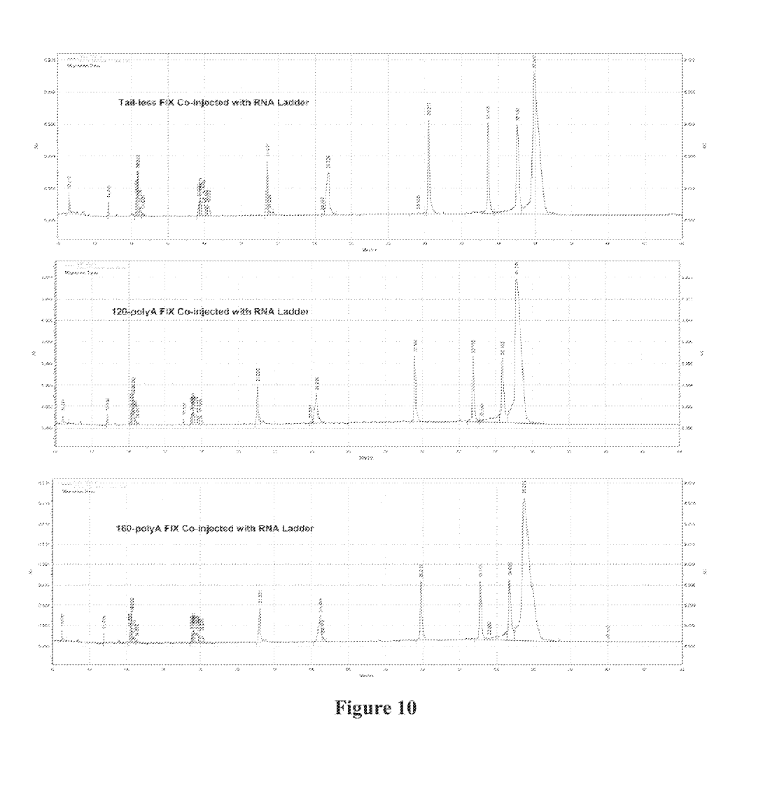

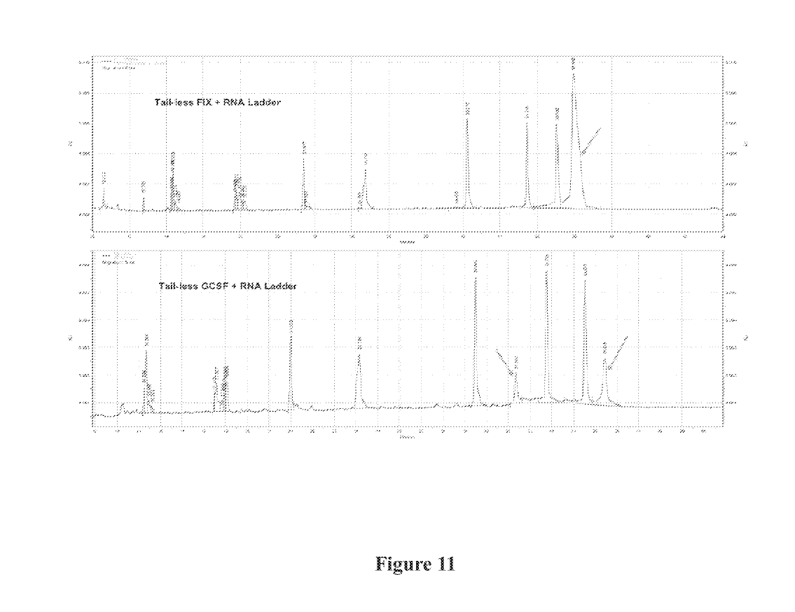

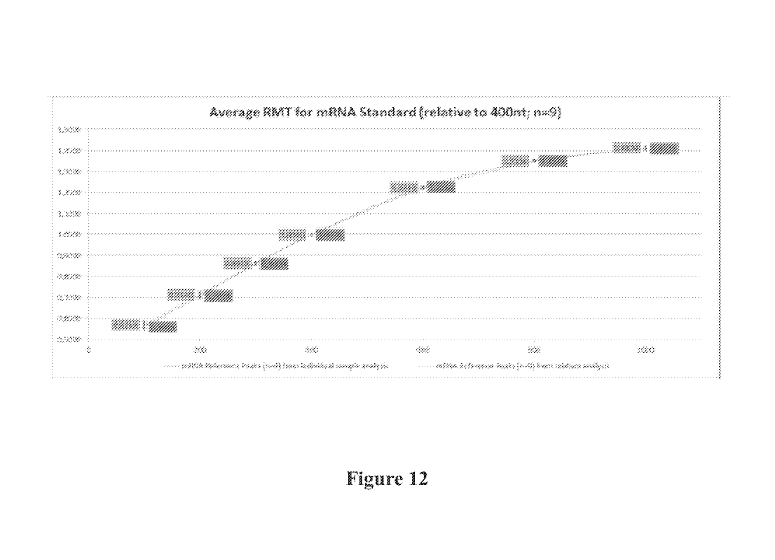

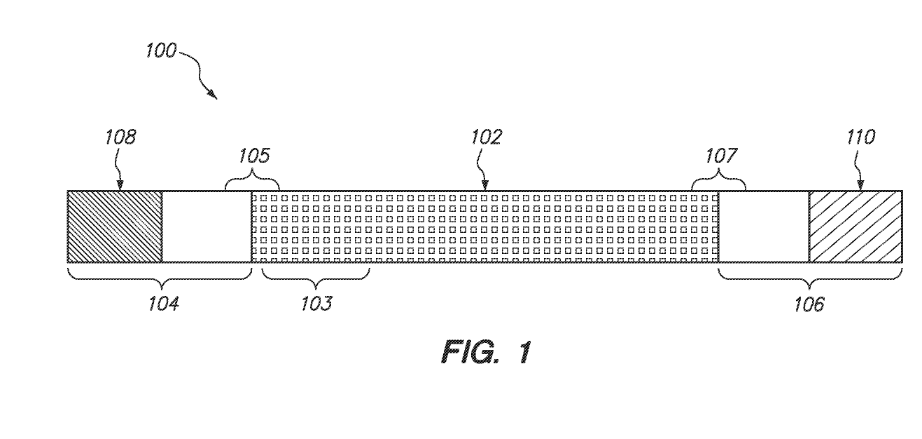

FIG. 1 shows a representative polynucleotide primary construct 100 of the present invention. As used herein, the term “primary construct” or “primary mRNA construct” refers to a polynucleotide transcript which encodes one or more polypeptides of interest and which retains sufficient structural and/or chemical features to allow the polypeptide of interest encoded therein to be translated. Primary constructs can be polynucleotides of the invention. When structurally or chemically modified, the primary construct can be referred to as an mmRNA (“modified mRNA”). Modified RNA, e.g., RNA transcripts, e.g., mRNA, are disclosed in the following which is incorporated by reference for all purposes: U.S. patent application Ser. No. 13/791,922, “Modified Polynucleotides for the Production of Biologics and Proteins Associated with Human Disease,” filed Mar. 9, 2013.

Returning to FIG. 1, the primary construct 100 here contains a first region of linked nucleotides 102 that is flanked by a first flanking region 104 and a second flaking region 106. As used herein, the “first region” can be referred to as a “coding region” or “region encoding” or simply the “first region.” This first region can include, but is not limited to, the encoded polypeptide of interest. The polypeptide of interest can comprise at its 5′ terminus one or more signal sequences encoded by a signal sequence region 103. The flanking region 104 can comprise a region of linked nucleotides comprising one or more complete or incomplete 5′ UTRs sequences. The flanking region 104 can also comprise a 5′ terminal cap 108. The second flanking region 106 can comprise a region of linked nucleotides comprising one or more complete or incomplete 3′ UTRs. The flanking region 106 can also comprise a 3′ tailing sequence 110.

Bridging the 5′ terminus of the first region 102 and the first flanking region 104 is a first operational region 105. Traditionally this operational region comprises a Start codon. The operational region can alternatively comprise any translation initiation sequence or signal including a Start codon.

Bridging the 3′ terminus of the first region 102 and the second flanking region 106 is a second operational region 107. Traditionally this operational region comprises a Stop codon. The operational region can alternatively comprise any translation initiation sequence or signal including a Stop codon. According to the present invention, multiple serial stop codons can also be used.

Generally, the shortest length of the first region of the primary construct of the present invention can be the length of a nucleic acid sequence that is sufficient to encode for a dipeptide, a tripeptide, a tetrapeptide, a pentapeptide, a hexapeptide, a heptapeptide, an octapeptide, a nonapeptide, or a decapeptide. In another embodiment, the length can be sufficient to encode a peptide of 2-30 amino acids, e.g. 5-30, 10-30, 2-25, 5-25, 10-25, or 10-20 amino acids. The length can be sufficient to encode for a peptide of at least 11, 12, 13, 14, 15, 17, 20, 25 or 30 amino acids, or a peptide that is no longer than 40 amino acids, e.g. no longer than 35, 30, 25, 20, 17, 15, 14, 13, 12, 11 or 10 amino acids. Examples of dipeptides that the polynucleotide sequences can encode or include, but are not limited to, carnosine and anserine.

Generally, the length of the first region encoding the polypeptide of interest of the present invention is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides). As used herein, the “first region” can be referred to as a “coding region” or “region encoding” or simply the “first region.”

In some embodiments, the polynucleotide, primary construct, or mmRNA includes from about 30 to about 100,000 nucleotides (e.g., from 100-10,000, from 600-10,000, from 700-3,000, from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

According to the present invention, the first and second flanking regions can range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

According to the present invention, the tailing sequence can range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length can be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

According to the present invention, the capping region can comprise a single cap or a series of nucleotides forming the cap. In this embodiment the capping region can be from 1 to 10, e.g. 2-9, 3-8, 4-7, 1-5, 5-10, or at least 2, or 10 or fewer nucleotides in length. In some embodiments, the cap is absent.

According to the present invention, the first and second operational regions can range from 3 to 40, e.g., 5-30, 10-20, 15, or at least 4, or 30 or fewer nucleotides in length and can comprise, in addition to a Start and/or Stop codon, one or more signal and/or restriction sequences.

### Flanking Regions: Untranslated Regions (UTRs)

Untranslated regions (UTRs) of a gene are transcribed but not translated. The 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides, primary constructs and/or mmRNA (“modified mRNA”) of the present invention to enhance the stability of the molecule. The specific features can also be incorporated to ensure controlled down-regulation of the transcript in case they are misdirected to undesired sites.

### 5′ UTR and Translation Initiation

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

By engineering the features typically found in abundantly expressed genes of specific target organs, one can enhance the stability and protein production of the polynucleotides, primary constructs or mmRNA of the invention. For example, introduction of 5′ UTR of liver-expressed mRNA, such as albumin, serum amyloid A, Apolipoprotein A/B/E, transferrin, alpha fetoprotein, erythropoietin, or Factor VIII, could be used to enhance expression of a nucleic acid molecule, such as a mmRNA, in hepatic cell lines or liver. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

Other non-UTR sequences can be incorporated into the 5′ (or 3′ UTR) UTRs. For example, introns or portions of introns sequences can be incorporated into the flanking regions of the polynucleotides, primary constructs or mmRNA of the invention. Incorporation of intronic sequences can increase protein production as well as mRNA levels.

### 3′ UTR and the AU Rich Elements

3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides, primary constructs or mmRNA of the invention. When engineering specific polynucleotides, primary constructs or mmRNA, one or more copies of an ARE can be introduced to make polynucleotides, primary constructs or mmRNA of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using polynucleotides, primary constructs or mmRNA of the invention and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

### 5′ Capping

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or anteterminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

Modifications to the polynucleotides, primary constructs, and mmRNA of the present invention can generate a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs can be chemically (i.e. non-enzymatically) or enzymatically synthesized and/or linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m7G(5′)ppp(5′)G). The 3′-O atom of the other, unmodified, guanine becomes linked to the 5′-terminal nucleotide of the Capped nucleic acid molecule (e.g. an mRNA or mmRNA). The N7- and 3′-O-methylated guanine provides the terminal moiety of the capped nucleic acid molecule (e.g. mRNA or mmRNA).

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides, primary constructs and mmRNA of the invention can also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those which, among other things, have enhanced binding of cap binding proteins, increased half life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

Because the polynucleotides, primary constructs or mmRNA can be capped post-transcriptionally, and because this process is more efficient, nearly 100% of the polynucleotides, primary constructs or mmRNA can be capped. This is in contrast to ˜80% when a cap analog is linked to an mRNA in the course of an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly-A Tails

During RNA processing, a long chain of adenine nucleotides (poly-A tail) can be added to a polynucleotide such as an mRNA molecules in order to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 100 and 250 residues long.

It has been discovered that unique poly-A tail lengths provide certain advantages to the polynucleotides, primary constructs or mmRNA of the present invention.

Generally, the length of a poly-A tail of the present invention is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the polynucleotide, primary construct, or mmRNA includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In one embodiment, the poly-A tail is designed relative to the length of the overall polynucleotides, primary constructs or mmRNA. This design can be based on the length of the coding region, the length of a particular feature or region (such as the first or flanking regions), or based on the length of the ultimate product expressed from the polynucleotides, primary constructs or mmRNA.

In this context the poly-A tail can be 10, 20, 30, 40, 50, 60, 70, 80, 90, or 100% greater in length than the polynucleotides, primary constructs or mmRNA or feature thereof. The poly-A tail can also be designed as a fraction of polynucleotides, primary constructs or mmRNA to which it belongs. In this context, the poly-A tail can be 10, 20, 30, 40, 50, 60, 70, 80, or 90% or more of the total length of the construct or the total length of the construct minus the poly-A tail. Further, engineered binding sites and conjugation of polynucleotides, primary constructs or mmRNA for Poly-A binding protein can enhance expression.

Additionally, multiple distinct polynucleotides, primary constructs or mmRNA can be linked together to the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In one embodiment, the polynucleotide primary constructs of the present invention are designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly-A tail. The resultant mmRNA construct is assayed for stability, protein production and other parameters including half-life at various time points. It has been discovered that the polyA-G quartet results in protein production equivalent to at least 75% of that seen using a poly-A tail of 120 nucleotides alone.

### Modifications

Herein, in a polynucleotide (such as a primary construct or an mRNA molecule), the terms “modification” or, as appropriate, “modified” refer to modification with respect to A, G, U or C ribonucleotides. Generally, herein, these terms are not intended to refer to the ribonucleotide modifications in naturally occurring 5′-terminal mRNA cap moieties. In a polypeptide, the term “modification” refers to a modification as compared to the canonical set of 20 amino acids, moiety).

The modifications can be various distinct modifications. In some embodiments, the coding region, the flanking regions and/or the terminal regions can contain one, two, or more (optionally different) nucleoside or nucleotide modifications. In some embodiments, a modified polynucleotide, primary construct, or mmRNA introduced to a cell can exhibit reduced degradation in the cell, as compared to an unmodified polynucleotide, primary construct, or mmRNA.

The polynucleotides, primary constructs, and mmRNA can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase can be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention can be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

As described herein, the polynucleotides, primary constructs, and mmRNA of the invention do not substantially induce an innate immune response of a cell into which the mRNA is introduced. Features of an induced innate immune response include 1) increased expression of pro-inflammatory cytokines, 2) activation of intracellular PRRs (RIG-I, MDA5, etc, and/or 3) termination or reduction in protein translation.

In certain embodiments, it can desirable to intracellularly degrade a modified nucleic acid molecule introduced into the cell. For example, degradation of a modified nucleic acid molecule can be preferable if precise timing of protein production is desired. Thus, in some embodiments, the invention provides a modified nucleic acid molecule containing a degradation domain, which is capable of being acted on in a directed manner within a cell. In another aspect, the present disclosure provides polynucleotides comprising a nucleoside or nucleotide that can disrupt the binding of a major groove interacting, e.g. binding, partner with the polynucleotide (e.g., where the modified nucleotide has decreased binding affinity to major groove interacting partner, as compared to an unmodified nucleotide).

The polynucleotides, primary constructs, and mmRNA can optionally include other agents (e.g., RNAi-inducing agents, RNAi agents, siRNAs, shRNAs, miRNAs, antisense RNAs, ribozymes, catalytic DNA, tRNA, RNAs that induce triple helix formation, aptamers, vectors, etc.). In some embodiments, the polynucleotides, primary constructs, or mmRNA can include one or more messenger RNAs (mRNAs) and one or more modified nucleoside or nucleotides (e.g., mmRNA molecules).

Design and Synthesis of mRNA

Polynucleotides, primary constructs or mmRNA for use in accordance with the invention can be prepared according to any available technique including, but not limited to chemical synthesis, enzymatic synthesis, which is generally termed in vitro transcription (IVT) or enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

The process of design and synthesis of the primary constructs of the invention generally includes the steps of gene construction, mRNA production (either with or without modifications) and purification. In the enzymatic synthesis method, a target polynucleotide sequence encoding the polypeptide of interest is first selected for incorporation into a vector which will be amplified to produce a cDNA template. Optionally, the target polynucleotide sequence and/or any flanking sequences can be codon optimized. The cDNA template is then used to produce mRNA through in vitro transcription (IVT). After production, the mRNA can undergo purification and clean-up processes.

### Vector Amplification

The vector containing the primary construct is then amplified and the plasmid isolated and purified using methods known in the art such as, but not limited to, a maxi prep using the Invitrogen PURELINK™ HiPure Maxiprep Kit (Carlsbad, Calif.).

### Plasmid Linearization

The plasmid can then be linearized using methods known in the art such as, but not limited to, the use of restriction enzymes and buffers. The linearization reaction can be purified using methods including, for example Invitrogen's PURELINK™ PCR Micro Kit (Carlsbad, Calif.), and HPLC based purification methods such as, but not limited to, strong anion exchange HPLC, weak anion exchange HPLC, reverse phase HPLC (RP-HPLC), and hydrophobic interaction HPLC (HIC-HPLC) and Invitrogen's standard PURELINK™ PCR Kit (Carlsbad, Calif.). The purification method can be modified depending on the size of the linearization reaction which was conducted. The linearized plasmid is then used to generate cDNA for in vitro transcription (IVT) reactions.

mRNA Production

The process of mRNA or mmRNA production can include, but is not limited to, in vitro transcription, cDNA template removal and RNA clean-up, and mRNA capping and/or tailing reactions.

### In Vitro Transcription

The cDNA produced in the previous step can be transcribed using an in vitro transcription (IVT) system. The system typically comprises a transcription buffer, nucleotide triphosphates (NTPs), an RNase inhibitor and a polymerase. The NTPs can be manufactured in house, can be selected from a supplier, or can be synthesized as described herein. The NTPs can be selected from, but are not limited to, those described herein including natural and unnatural (modified) NTPs. The polymerase can be selected from, but is not limited to, T7 RNA polymerase, T3 RNA polymerase and mutant polymerases such as, but not limited to, polymerases able to incorporate modified nucleic acids.

### RNA Polymerases

Any number of RNA polymerases or variants can be used in the design of the primary constructs of the present invention.

RNA polymerases can be modified by inserting or deleting amino acids of the RNA polymerase sequence. As a non-limiting example, the RNA polymerase can be modified to exhibit an increased ability to incorporate a 2′-modified nucleotide triphosphate compared to an unmodified RNA polymerase (see International Publication WO2008078180 and U.S. Pat. No. 8,101,385; herein incorporated by reference in their entireties).

Variants can be obtained by evolving an RNA polymerase, optimizing the RNA polymerase amino acid and/or nucleic acid sequence and/or by using other methods known in the art. As a non-limiting example, T7 RNA polymerase variants can be evolved using the continuous directed evolution system set out by Esvelt et al. (Nature (2011) 472(7344):499-503; herein incorporated by reference in its entirety) where clones of T7 RNA polymerase can encode at least one mutation such as, but not limited to, lysine at position 93 substituted for threonine (K93T), I4M, A7T, E63V, V64D, A65E, D66Y, T76N, C125R, S128R, A136T, N165S, G175R, H176L, Y178H, F182L, L196F, G198V, D208Y, E222K, S228A, Q239R, T243N, G259D, M267I, G280C, H300R, D351A, A354S, E356D, L360P, A383V, Y385C, D388Y, S397R, M401T, N410S, K450R, P451T, G452V, E484A, H523L, H524N, G542V, E565K, K577E, K577M, N601S, S684Y, L699I, K713E, N748D, Q754R, E775K, A827V, D851N or L864F. As another non-limiting example, T7 RNA polymerase variants can encode at least mutation as described in U.S. Pub. Nos. 20100120024 and 20070117112; herein incorporated by reference in their entireties. Variants of RNA polymerase can also include, but are not limited to, substitutional variants, conservative amino acid substitution, insertional variants, deletional variants and/or covalent derivatives.

In one embodiment, the primary construct can be designed to be recognized by the wild type or variant RNA polymerases. In doing so, the primary construct can be modified to contain sites or regions of sequence changes from the wild type or parent primary construct.

In one embodiment, the primary construct can be designed to include at least one substitution and/or insertion upstream of an RNA polymerase binding or recognition site, downstream of the RNA polymerase binding or recognition site, upstream of the TATA box sequence, downstream of the TATA box sequence of the primary construct but upstream of the coding region of the primary construct, within the 5′UTR, before the 5′UTR and/or after the 5′UTR.

In one embodiment, the 5′UTR of the primary construct can be replaced by the insertion of at least one region and/or string of nucleotides of the same base. The region and/or string of nucleotides can include, but is not limited to, at least 3, at least 4, at least 5, at least 6, at least 7 or at least 8 nucleotides and the nucleotides can be natural and/or unnatural. As a non-limiting example, the group of nucleotides can include 5-8 adenine, cytosine, thymine, a string of any of the other nucleotides disclosed herein and/or combinations thereof.

In one embodiment, the 5′UTR of the primary construct can be replaced by the insertion of at least two regions and/or strings of nucleotides of two different bases such as, but not limited to, adenine, cytosine, thymine, any of the other nucleotides disclosed herein and/or combinations thereof. For example, the 5′UTR can be replaced by inserting 5-8 adenine bases followed by the insertion of 5-8 cytosine bases. In another example, the 5′UTR can be replaced by inserting 5-8 cytosine bases followed by the insertion of 5-8 adenine bases.

In one embodiment, the primary construct can include at least one substitution and/or insertion downstream of the transcription start site which can be recognized by an RNA polymerase. As a non-limiting example, at least one substitution and/or insertion can occur downstream the transcription start site by substituting at least one nucleic acid in the region just downstream of the transcription start site (such as, but not limited to, +1 to +6). Changes to region of nucleotides just downstream of the transcription start site can affect initiation rates, increase apparent nucleotide triphosphate (NTP) reaction constant values, and increase the dissociation of short transcripts from the transcription complex curing initial transcription (Brieba et al., Biochemistry (2002) 41: 5144-5149; herein incorporated by reference in its entirety). The modification, substitution and/or insertion of at least one nucleic acid can cause a silent mutation of the nucleic acid sequence or can cause a mutation in the amino acid sequence.

In one embodiment, the primary construct can include the substitution of at least 1, at least 2, at least 3, at least 4, at least 5, at least 6, at least 7, at least 8, at least 9, at least 10, at least 11, at least 12 or at least 13 guanine bases downstream of the transcription start site.

In one embodiment, the primary construct can include the substitution of at least 1, at least 2, at least 3, at least 4, at least 5 or at least 6 guanine bases in the region just downstream of the transcription start site. As a non-limiting example, if the nucleotides in the region are GGGAGA the guanine bases can be substituted by at least 1, at least 2, at least 3 or at least 4 adenine nucleotides. In another non-limiting example, if the nucleotides in the region are GGGAGA the guanine bases can be substituted by at least 1, at least 2, at least 3 or at least 4 cytosine bases. In another non-limiting example, if the nucleotides in the region are GGGAGA the guanine bases can be substituted by at least 1, at least 2, at least 3 or at least 4 thymine, and/or any of the nucleotides described herein.

In one embodiment, the primary construct can include at least one substitution and/or insertion upstream of the start codon. For the purpose of clarity, one of skill in the art would appreciate that the start codon is the first codon of the protein coding region whereas the transcription start site is the site where transcription begins. The primary construct can include, but is not limited to, at least 1, at least 2, at least 3, at least 4, at least 5, at least 6, at least 7 or at least 8 substitutions and/or insertions of nucleotide bases. The nucleotide bases can be inserted or substituted at 1, at least 1, at least 2, at least 3, at least 4 or at least 5 locations upstream of the start codon. The nucleotides inserted and/or substituted can be the same base (e.g., all A or all C or all T or all G), two different bases (e.g., A and C, A and T, or C and T), three different bases (e.g., A, C and T or A, C and T) or at least four different bases. As a non-limiting example, the guanine base upstream of the coding region in the primary construct can be substituted with adenine, cytosine, thymine, or any of the nucleotides described herein. In another non-limiting example the substitution of guanine bases in the primary construct can be designed so as to leave one guanine base in the region downstream of the transcription start site and before the start codon (see Esvelt et al. Nature (2011) 472(7344):499-503; herein incorporated by reference in its entirety). As a non-limiting example, at least 5 nucleotides can be inserted at 1 location downstream of the transcription start site but upstream of the start codon and the at least 5 nucleotides can be the same base type.

cDNA Template Removal and Clean-Up

The cDNA template can be removed using methods known in the art such as, but not limited to, treatment with Deoxyribonuclease I (DNase I). RNA clean-up can also include a purification method such as, but not limited to, AGENCOURT® CLEANSEQ® system from Beckman Coulter (Danvers, Mass.), HPLC based purification methods such as, but not limited to, strong anion exchange HPLC, weak anion exchange HPLC, reverse phase HPLC (RP-HPLC), and hydrophobic interaction HPLC (HIC-HPLC).

Capping and/or Tailing Reactions

The primary construct or mmRNA can also undergo capping and/or tailing reactions. A capping reaction can be performed by methods known in the art to add a 5′ cap to the 5′ end of the primary construct. Methods for capping include, but are not limited to, using a Vaccinia Capping enzyme (New England Biolabs, Ipswich, Mass.).

A poly-A tailing reaction can be performed by methods known in the art, such as, but not limited to, 2′ O-methyltransferase and by methods as described herein. If the primary construct generated from cDNA does not include a poly-T, it can be beneficial to perform the poly-A-tailing reaction before the primary construct is cleaned.

mRNA Characterization and Purification

Primary construct or mmRNA purification can include, but is not limited to, mRNA or mmRNA clean-up, quality assurance and quality control. mRNA or mmRNA clean-up can be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or chromatography based purification methods such as, but not limited to, strong anion exchange HPLC, weak anion exchange HPLC, reverse phase HPLC (RP-HPLC), hydrophobic interaction HPLC (HIC-HPLC), size exclusion chromatography, and ion pairing chromatography. The term “purified” when used in relation to a polynucleotide such as a “purified mRNA or mmRNA” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

A quality assurance and/or quality control check can be conducted using methods such as, but not limited to, gel electrophoresis, UV absorbance, capillary electrophoresis, capillary gel electrophoresis, analytical HPLC, or mass spectrometry.

In another embodiment, the mRNA or mmRNA can be sequenced by methods including, but not limited to reverse-transcriptase-PCR.

In one embodiment, the mRNA or mmRNA can be quantified using methods such as, but not limited to, ultraviolet visible spectroscopy (UV/Vis). A non-limiting example of a UV/Vis spectrometer is a NANODROP® spectrometer (ThermoFisher, Waltham, Mass.). The quantified mRNA or mmRNA can be analyzed in order to determine if the mRNA or mmRNA is of proper size, and to check that no major fragmentation of the mRNA or mmRNA has occurred, which might affect the extinction coefficient. Degradation of the mRNA and/or mmRNA can be checked by methods such as, but not limited to, agarose gel electrophoresis, HPLC based methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE), capillary gel electrophoresis (CGE), size exclusion chromatography, and ion-pairing chromatography.

### Sequencing Methods/Reverse Transcriptase Sequencing

The Sanger method is a common method for DNA sequencing. Reverse transcription coupled to the Sanger method is possible to determine RNA sequences.

In one embodiment of the invention, a reverse transcription reaction is performed with an RNA transcript, a reverse transcriptase, deoxyribonucleotides, and a set of primers. In some embodiments, the set of primers include several closely spaced forward and reverse primers. The use of several primers makes it possible to work with large mRNAs. The primers are a combination of internal mRNA sequence specific primers, and primers found in the untranslated regions of mRNA. In further embodiments, the reverse transcription reaction results in several cDNA molecules that cover the length of the RNA transcript.

In another embodiment of the invention, the cDNA product or products from the reverse transcription reaction are incubated with a set of primers under conditions sufficient to allow PCR (polymerase chain reaction) to occur. The use of several primers makes it possible to work with large mRNAs. The primers are a combination of internal mRNA sequence specific primers, and primers found in the untranslated regions of mRNA. In some embodiments, the set of primers include several closely spaced forward and reverse primers.

The amplified cDNA molecules can then be analyzed using a DNA sequencing method. In one embodiment, the DNA sequencing method is the Sanger method. Other sequencing methods include CAGE tag-sequencing (Hoon 2008), deep sequencing, bidirectional sequencing, RNA sequencing, shotgun sequencing, bridge PCR, massively parallel signature sequencing (MPSS), polony sequencing, pyrosequencing, Illumina (Solexa) sequencing SOLiD sequencing, ion semiconductor sequencing, DNA nanoball sequencing, heliscope single molecule sequencing, and single molecule real time (SMRT) sequencing. By sequencing the cDNA, the sequence of the RNA transcript can be determined.

### Oligonucleotide Mapping

Oligonucleotide mapping involves incubating an RNA transcript with multiple polynucleotide (DNA or RNA) probes under conditions allowing the hybridization of the probes to the RNA transcript to form duplexes along different regions of the RNA transcript. In one embodiment, the probes are antisense probes. In another embodiment, the probes are 10-40 nucleotides in length. In a further embodiment, the probes are 15-30 nucleotides in length. In another embodiment, the probes are less than about 20 nucleotides in length, and at least 8 of those nucleotides are deoxyribonucleotides. In another embodiment, the probes are complementary to the 3′ end of the RNA immediately upstream of the poly-A tail. The probes can be dispersed throughout the RNA transcript, binding at regions less than about 50 nucleotides apart on the RNA transcript. After the duplexes have formed, an RNase is added under conditions sufficient to allow the RNase to digest portions of the duplexes along the RNA transcript, forming RNA fragments. The RNase can be any RNase that can cleave such duplexes, such as RNase H or RNase T1. The fragment mRNA can then be characterized by HPLC coupled to mass spectrometry (MS).

### Mass Spectrometry

Mass spectrometry (MS) is an analytical technique that can provide structural and molecular mass/concentration information on molecules after their conversion to ions. The molecules are first ionized to acquire positive or negative charges and then they travel through the mass analyzer to arrive at different areas of the detector according to their mass/charge (m/z) ratio. The sequence and composition of small oligonucleotides (<30 nt) can be determined by LC/MS/MS (liquid chromatography-tandem mass spectrometry) or RT-qPCR sequencing. In the literature, analysis of up to 250 nt for DNA by mass spectrometry is reported, but not for RNA larger than 1000 nt. For example, the group of Oberacher et al. published several papers on the mass spectrometry analysis of PCR products. Oberacher H, Pitterl F. On the use of ESI-QqTOE-MS/MS for the comparative sequencing of nucleic acids. Biopolymers. 2009 June; 91(6):401-9.

Mass spectrometry of large mRNA has been difficult since a) the mRNA is highly charged and generates a broad mass envelop in electrospray-MS that is difficult to deconvolute, b) it is difficult to differentiate missing or modifications in one or a few nucleotides out of hundreds to thousands of nucleotides.

Mass spectrometry is performed using a mass spectrometer which includes an ion source for ionizing the fractionated sample and creating charged molecules for further analysis. For example ionization of the sample can be performed by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. The skilled artisan will understand that the choice of ionization method can be determined based on the analyte to be measured, type of sample, the type of detector, the choice of positive versus negative mode, etc.

After the sample has been ionized, the negatively charged ions thereby created can be analyzed to determine a mass-to-charge ratio (i.e., m/z). Suitable analyzers for determining mass-to-charge ratios include quadropole analyzers, ion traps analyzers, and time-of-flight analyzers. The ions can be detected using several detection modes. For example, selected ions can be detected (i.e., using a selective ion monitoring mode (SIM)), or alternatively, ions can be detected using a scanning mode, e.g., multiple reaction monitoring (MRM) or selected reaction monitoring (SRM).

### Anion Exchange HPLC

Anion exchange (AEX) chromatography is a method of purification and analysis that leverages ionic interaction between positively charged sorbents and negatively charged molecules. AEX sorbents consist of a charged functional group (e.g. quaternary amine, polyethylenimine, diethylaminoethyl, dimethylaminopropyl etc.), cross-linked to solid phase media. There are two categories of anion exchange media, “strong” and “weak” exchangers. Strong exchangers maintain a positive charge over a broad pH range, while weak exchangers only exhibit charge over a specific pH range. Anion exchange resins facilitate RNA capture due to the interaction with the negatively charged phosphate backbone of the RNA providing an ideal mode of separation.

The mechanism of purification or analysis can involve binding the RNA under relatively low ionic strength solution to an AEX sorbent. Loading conditions for the AEX chromatography can be performed under non-denaturing conditions with or without the addition of chaotropic salts as well as loading under denaturing conditions which can or can not include the use of chaotropic agents. Thermal and chemical denaturation is the preferred method of denaturing the RNA for analytical purposes.

AEX chromatography materials can include weak resins. Weak resins include resins that have a low affinity for polypeptides and a high affinity for polynucleotides, e.g., RNA transcripts. Furthermore, weak resins also include resins that have a low affinity for polypeptides and a low affinity for polynucleotides, e.g., RNA transcripts. AEX chromatography materials can also include porous IEX media: polystyrene divinylbenzene, polymethacrylate, crosslinked agarose, allyl dextran/N-N-bis acylamide, or silica, for example. In one embodiment, non-porous IEX media such as monolithic columns can be used. In another embodiment, membrane-based ion exchangers are used, including Millipore chromasorb and Sartorius sartobind. In some embodiments, AEX chromatography conditions can include strong or weak anion exchange groups, mixed mode, heated or unheated conditions, denaturing or non-denaturing conditions, various particle/pore sizes, pH range between 3 and 9. In other embodiments, chaotropic salts are used, such as urea, perchlorate, and guanidinium salts. Examples of mobile phase compositions include the entire hofmeister seris of ions, salts such as chlorine, bromine, citrate, iodide, sulfate, phosphate, perchlorate, and counter-ions/cations such as sodium, potassium, and calcium. Additives/modifiers to the mobile phase can include organics such as ethanol, acetonitrile, and IPA. In some embodiments, the buffer comprises Tris, HEPES, or phosphate.

In some embodiments of the invention, the RNA transcript is denatured before undergoing AEX, using >6 M urea (preferably >7 M urea), and heating the RNA transcript to >70° Celsius for about 5 minutes. The RNA transcript is then contacted with an ion exchange sorbent with a positively-charged functional group linked to solid phase media. The RNA transcript sample is delivered with a mobile phase, so that the RNA transcript binds the positively-charged functional group of the ion exchange sorbent. In one embodiment, the mobile phase is a Tris-EDTA-acetonitrile buffered mobile phase. In another embodiment, there are two mobile phases that include Tris-EDTA-acetonitrile buffer. In a further embodiment, the mobile phase contains a strong chaotropic salt, such as sodium perchlorate. Next, the RNA transcript and any impurities are eluted from the ion exchange sorbent. The RNA transcript and any impurities are then analyzed. The analysis can include analysis of charge heterogeneity of the RNA transcript, mass heterogeneity of the RNA transcript, process intermediates, hybridization impurities, and degradation products.

### Capillary Electrophoresis

Capillary gel electrophoresis (CGE) separates molecules based on molecular weight and charge. To prepare CGE, a gel is delivered into a capillary with an electrolyte medium. An RNA sample can then be delivered into the capillary. An electric field is applied to the capillary that causes the RNA transcript to migrate through the capillary. The RNA transcript has a different electrophoretic mobility than any impurities in the sample, so the RNA transcript migrates through the capillary at the rate that is different from the rate at which the impurities migrate through the capillary, since the impurities are smaller and therefore less charged than the RNA transcript. In one embodiment, the electrophoretic mobility of the RNA transcript is proportional to mass and ionic charge of the RNA transcript, and inversely proportional to frictional forces in the electrolyte medium. In another embodiment, the sample is delivered under denaturing conditions. The CGE allows for analysis of the charge heterogeneity of the RNA transcript and the impurities.

### Detection of RNA Impurities

The detection of RNA impurities involves the detection of smaller molecular weight and hybridized RNA molecules in a sample comprising the RNA transcript. This includes RNA-RNA and RNA-DNA hybrids.

In some embodiments, detecting shorter mRNA transcripts includes denaturing the RNA transcript, and subjecting the denatured RNA transcript to HPLC analysis, where the HPLC separates and quantifies any short mRNA transcript impurities. In further embodiments, the HPLC is reverse phase HPLC, and the method is coupled to tandem mass spectrometry to further characterize the impurities.

In other embodiments, the RNA transcript is further filtered, and collected as retentate (e.g., 50 kDa molecular weight cut-off filter), whereas the filtrate contains the impurities and is characterized by mass spectrometry.

In one embodiment, detecting the RNA-RNA and RNA-DNA hybrid impurities includes treating an RNA transcript with urea in the presence of EDTA, and subjecting the treated RNA transcript to spin filtration, where the retentate retains the RNA transcript and the filtrate collects the impurities. The filtrate impurities can be analyzed using anion exchange-HPLC, ion pair reverse phase HPLC, or electrospray ionization mass spectrometry, among other methods.

### EXAMPLES

### Example 1

### Preparing Plasmids for cDNA Production

cDNA is produced to provide a DNA template for in vitro transcription. To prepare plasmids for producing cDNA, NEB DH5-alpha competent E. coli are used in one example. Transformations are performed according to NEB instructions using 100 ng of plasmid. The protocol is as follows:

Spread 50-100 μl of each dilution onto a selection plate and incubate overnight at 37° C. Alternatively, incubate at 30° C. for 24-36 hours or 25° C. for 48 hours.

A single colony is then used to inoculate 5 ml of LB growth media using the appropriate antibiotic and then allowed to grow (250 RPM, 37° C.) for 5 hours. This is then used to inoculate a 200 ml culture medium and allowed to grow overnight under the same conditions.

To isolate the plasmid (up to 850 μg), a maxi prep is performed using the Invitrogen PURELINK™ HiPure Maxiprep Kit (Carlsbad, Calif.), following the manufacturer's instructions, which are as follows: thaw a tube of NEB 5-alpha Competent E. coli cells on ice for 10 minutes. Add 1-5 μl containing 1 pg-100 ng of plasmid DNA to the cell mixture. Carefully flick the tube 4-5 times to mix cells and DNA. Do not vortex. Place the mixture on ice for 30 minutes. Do not mix. Heat shock at 42° C. for 30 seconds. Do not mix. Place on ice for 5 minutes. Do not mix. Pipette 950 μl of room temperature SOC into the mixture. Place at 37° C. for 60 minutes. Shake vigorously (250 rpm) or rotate. Warm selection plates to 37° C. Mix the cells thoroughly by flicking the tube and inverting.

In order to generate cDNA for in vitro transcription (IVT), the plasmid is first linearized using a restriction enzyme such as XbaI. A typical restriction digest with XbaI will comprise the following: Plasmid 1.0 μg; 10× Buffer 1.0 μl; XbaI 1.5 μl; dH₂0 up to 10 μl; incubated at 37° C. for 1 hr. If performing at lab scale (<5 μg), the reaction is cleaned up using Invitrogen's PURELINK™ PCR Micro Kit (Carlsbad, Calif.) per manufacturer's instructions. Larger scale purifications can need to be done with a product that has a larger load capacity such as Invitrogen's standard PURELINK™ PCR Kit (Carlsbad, Calif.). Following the cleanup, the linearized vector is quantified using the NanoDrop and analyzed to confirm linearization using agarose gel electrophoresis.

### Example 2

### PCR for cDNA Production

PCR procedures for the preparation of cDNA are performed using 2×KAPA HIFI™ HotStart ReadyMix by Kapa Biosystems (Woburn, Mass.). This system includes 2×KAPA ReadyMix12.5 μl; Forward Primer (10 uM) 0.75 μl; Reverse Primer (10 uM) 0.75 μl; Template cDNA 100 ng; and dH₂0 diluted to 25.0 μl. The reaction conditions are at 95° C. for 5 min. and 25 cycles of 98° C. for 20 sec, then 58° C. for 15 sec, then 72° C. for 45 sec, then 72° C. for 5 min. then 4° C. to termination.

The reverse primer of the instant invention incorporates a poly-T₁₂₀ for a poly-A₁₂₀ in the mRNA. Other reverse primers with longer or shorter poly(T) tracts can be used to adjust the length of the poly(A) tail in the mRNA.

The reaction is cleaned up using Invitrogen's PURELINK™ PCR Micro Kit (Carlsbad, Calif.) per manufacturer's instructions (up to 5 μg). Larger reactions will require a cleanup using a product with a larger capacity. Following the cleanup, the cDNA is quantified using the NANODROP™ and analyzed by agarose gel electrophoresis to confirm the cDNA is the expected size. The cDNA is then submitted for sequencing analysis before proceeding to the in vitro transcription reaction.

### Example 3

### In Vitro Transcription (IVT)

mRNAs according to the invention can be made using standard laboratory methods and materials. The open reading frame (ORF) of the gene of interest can be flanked by a 5′ untranslated region (UTR), which can contain a strong Kozak translational initiation signal and/or an alpha-globin 3′ UTR which can include an oligo(dT) sequence for templated addition of a poly-A tail. The mRNAs can be modified to reduce the cellular innate immune response. The modifications to reduce the cellular response can include pseudouridine (ψ) and 5-methyl-cytidine (5meC, 5mc or m⁵C). (See, Kariko K et al. Immunity 23:165-75 (2005), Kariko K et al. Mol Ther 16:1833-40 (2008), Anderson B R et al. NAR (2010); each of which are herein incorporated by reference in their entireties).

The ORF can also include various upstream or downstream additions (such as, but not limited to, β-globin, tags, etc.) can be ordered from an optimization service such as, but limited to, DNA2.0 (Menlo Park, Calif.) and can contain multiple cloning sites which can have XbaI recognition. Upon receipt of the construct, it can be reconstituted and transformed into chemically competent E. coli.

The in vitro transcription reaction can generate mRNA containing modified nucleotides or modified RNA. The input nucleotide triphosphate (NTP) mix is made in-house using natural and un-natural NTPs.

A typical in vitro transcription reaction includes the following:

| | | |
|---|---|---|
| 1 | Template cDNA | 1.0 μg |
| 2 | 10x transcription buffer | 2.0 μl |
| | (400 mM Tris-HCl pH 8.0, | |
| | 190 mM MgCl₂, 50 mM DTT, | |
| | 10 mM Spermidine) | |
| 3 | Custom NTPs (25 mM each) | 7.2 μl |
| 4 | RNase Inhibitor | 20U |
| 5 | T7 RNA polymerase | 3000U |
| 6 | dH₂0 | Up to 20.0 μl. and |
| 7 | Incubation at 37° C. for 3 hr-5 hrs. | |

The crude IVT mix can be stored at 4° C. overnight for cleanup the next day. 1 U of RNase-free DNase is then used to digest the original template. After 15 minutes of incubation at 37° C., the mRNA is purified using Ambion's MEGACLEAR™ Kit (Austin, Tex.) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred. 3′

### Example 4

### Oligonucleotide Mapping

We have designed a method to generate site-specific cleavage of mRNA into discrete fragments that are then characterized by mass spectrometry. Our approach was to design short cDNA molecules (e.g. 30-40 nucleotides) at target sites along the mRNA sequence, anneal the antisense to the mRNA, and subsequently use RNaseH to digest into specific sites. The resulting RNA fragments are separated by either anion exchange HPLC (AEX) or RP-HPLC, where the latter can be characterized by mass spectrometry. RNase H binds to double stranded DNA/RNA hybrids and cleaves the mRNA in the hybridized sequence. As an example proof of concept, two antisense molecules were synthesized to target characterization of the polyA tail of GCSF mRNA: antisense strand 1 was partially complementary to the poly-A-tail and the cleavage site was expected to be directly adjacent to the 5′-end of the poly-A-tail after position 759 of GCSF: 3′-ucaucdCdTdTdCdTdTdTdTuuuuu-5′. Antisense 2 is complementary to the 18 bases directly 5′-adjacent to the poly-A-tail after position 750: 3′-uucggdAdCdTdCdAdTdCdCuucuu-5′.

In the presence of a polyA tail, antisense 2 will bind near the 3′-tail and cleavage of the mRNA will occur within the duplex, releasing the polyA tail as well as some additional nucleotides. Sample preparation involves first a heating/cooling step to partially open the secondary structures of the mRNA and allow annealing of antisense strand to the mRNA. 10 mM EDTA was added prior to the annealing step since any divalent cations would lead to mRNA cleavage. Then antisense is added and heated for 3 min at 90 C, then cooled to 37° C. for hybridizing to the mRNA. The hybridized or non-hybridized mRNA were then incubated with 1 Unit of RNase H in 100 μL of RNase H reaction buffer (50 mM Tris-HCl, 75 mM KCl, 3 mM MgCl₂, 10 mM DTT, at pH 8.3 @ 25° C., available as a ready-to-use solution from New England Biolabs) at 37° C. for 20-30 minutes in the presence of MgCl2 (10 mM) to get at least 3 molar ratio of Mg to enzyme. RNase H is then inhibited by addition of excess EDTA. Samples were then analysed by AEX and RP-HPLC.

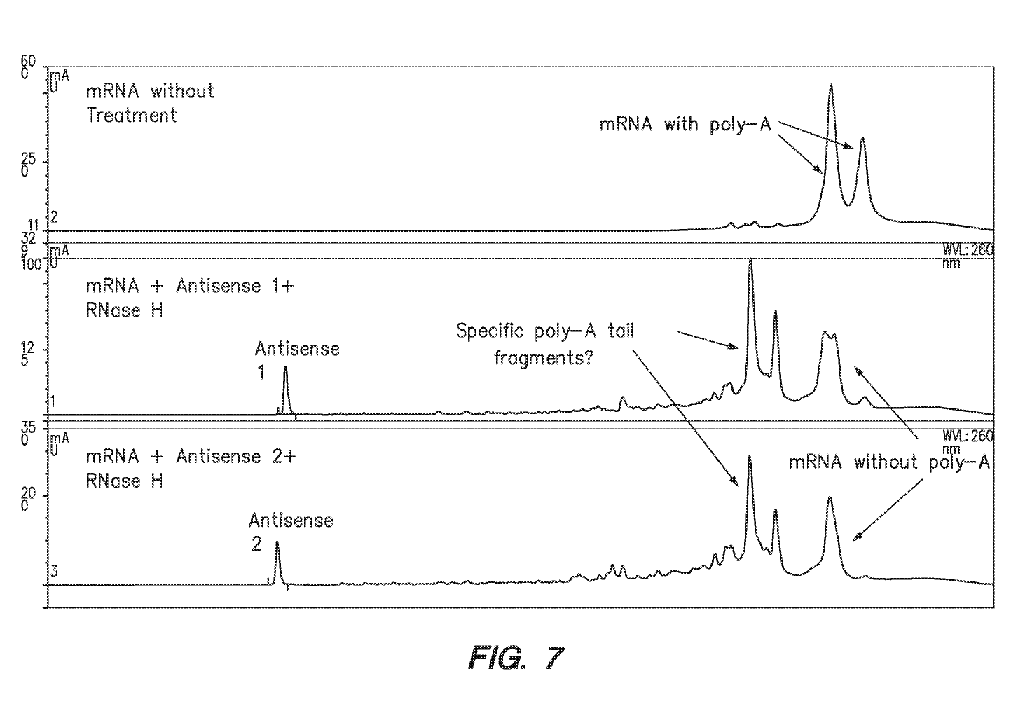

Table 4 (below) provides the running conditions for AEX analysis. FIG. 7, top shows the AEX chromatogram of a GCSF mRNA containing 899 nucleotides including ˜140 polyA at the 3′end. The identity of the two peaks is not known, however, both peaks contain polyA tail as the batch has been purified by oligodT affinity chromatography. Following antisense annealing and RNAse digestion, two major earlier eluting peaks are observed (FIG. 7, middle and bottom). These species can represent the polyA tail portion after site-specific cleavage of the mRNA. The hybridized antisense results in RNase H cleavage of the polyA tail. In addition, a peak co-eluting with the position of mRNA lacking polyA (from previous experiments using CE as described below) is observed. Filtration of the samples through ≦50 kDa membrane enables characterization of the smaller 3′-tail polynucleotides in the filtrate by LC/MS without interference from the remaining larger 5′-end mRNA fragment in the retentate.

### Example 5

### Reverse Transcriptase Sequencing

To verify the fidelity of the in vitro transcription reaction, the sequence of the original plasmid, the PCR product, and the final manufactured mRNA was determined. Linearized plasmid was amplified and sequenced by the Sanger method using primers CP1, CP2, CP3 and CP4

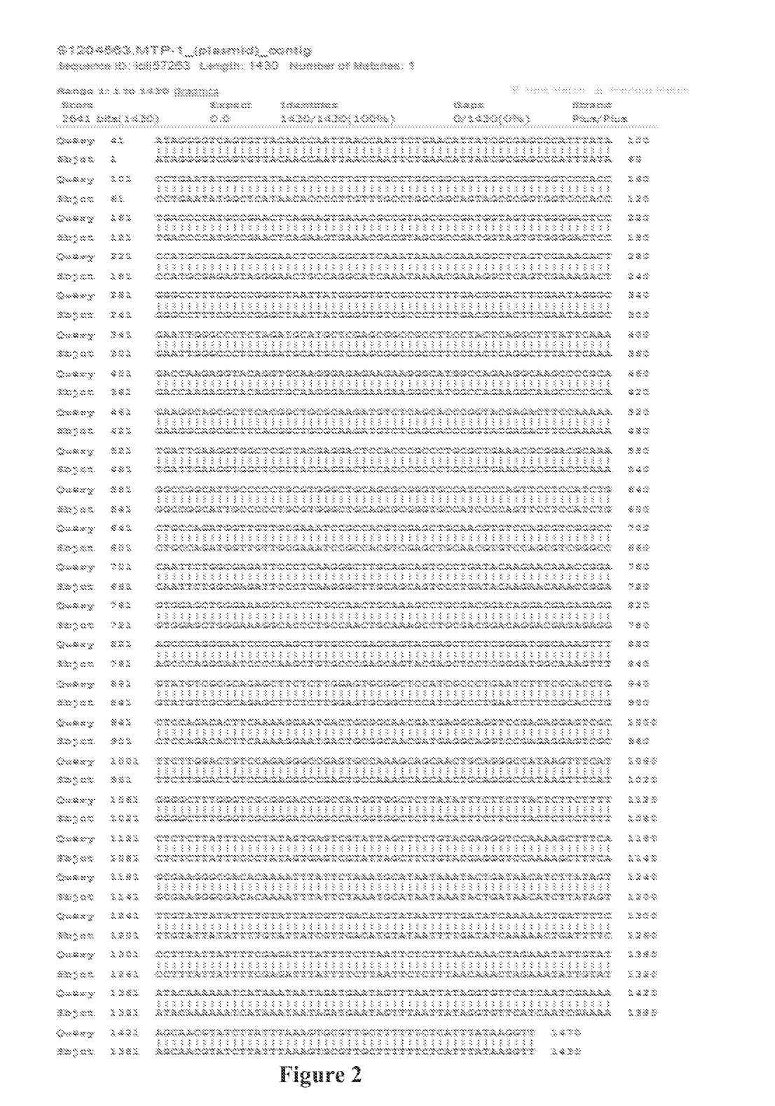

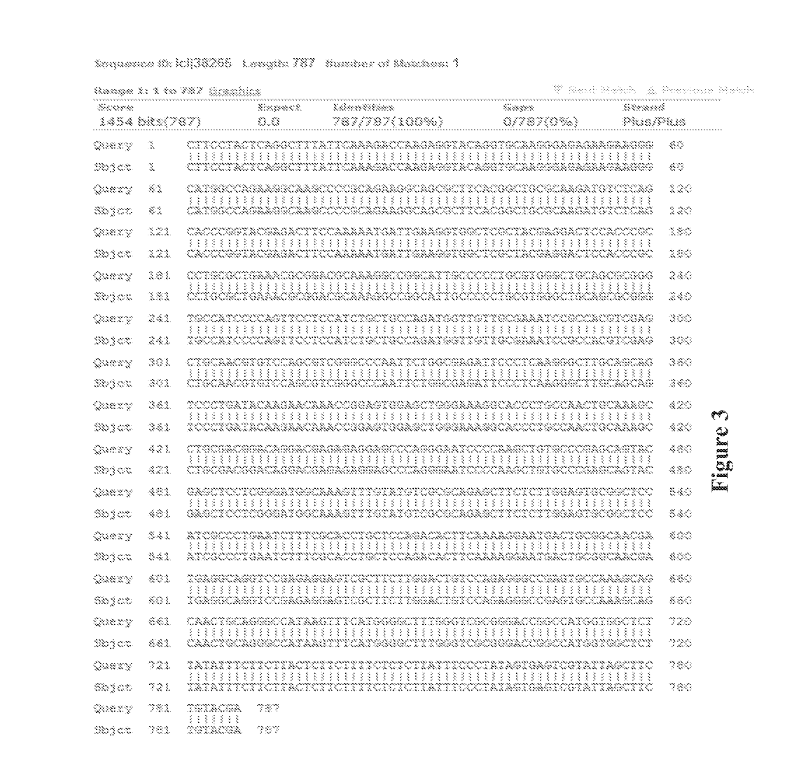

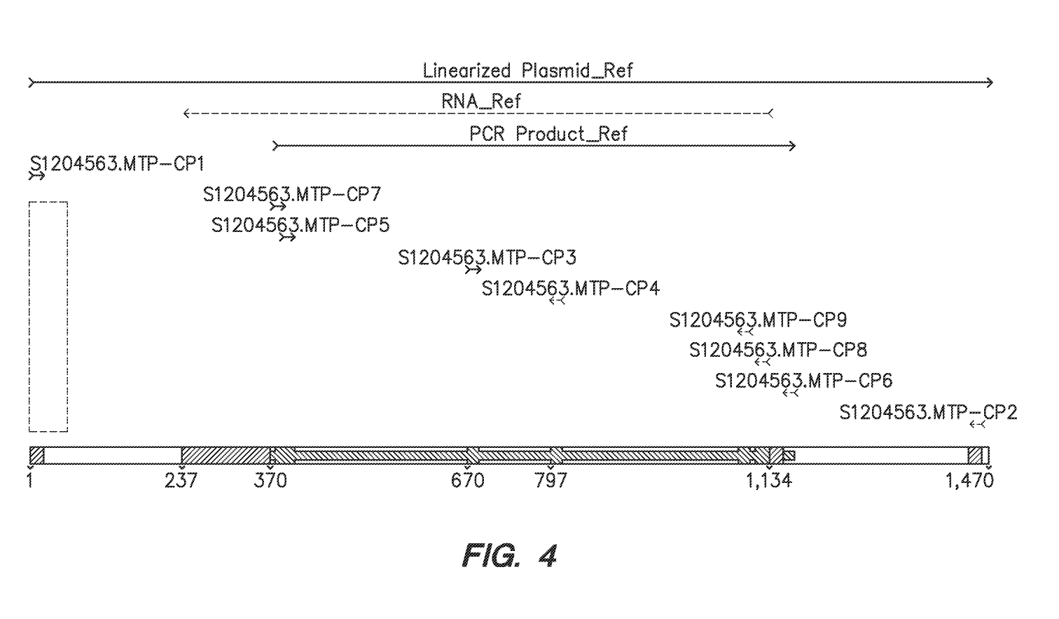

(Table 1). PCR product was amplified and sequenced using primers CP5 and CP6. The PCR program conditions used to amplify DNA template is described in Table 2. Bidirectional sequencing results were achieved for each PCR amplified sample template using fluorescent dye-terminator chemistry and ABI Prism™ 3730xl DNA sequencers, which typically give >650 bp Q20/Phred20 read lengths. Using these methodologies, the sequence of the linearized plasmid and PCR product exhibited 100% identity with 97% and 98% coverage, respectively (FIG. 2 and FIG. 3).

TABLE 1
Primer Sequences Used for
Plasmid, PCR Product, and mRNA Sequencing

| Primer Name | Primer Sequence |
|---|---|
| S1300312.MTP-CP1 | CATTCAAATATGTATCCGCTC |
| S1300312.MTP-CP2 | GAGAAAAAAGCAACGCAC |
| S1300312.MTP-CP3 | CGTCGAGCTGCAACGTG |
| S1300312.MTP-CP4 | CGTCCTGTCCGTCGCAG |
| S1300312.MTP-CP5 | CAGGCTTTATTCAAAGAC |
| S1300312.MTP-CP6 | GGACCCTCGTACAGAAG |
| S1300312.MTP-CP7 | TTTTTTTCTTCCTACTCAGGC |
| S1300312.MTP-CP8 | GGAAATAAGAGAGAAAAGAAGAG |
| S1300312.MTP-CP9 | GAAATATAAGAGCCACCATGG |
| S1300312.MTP-CP10 | CTCTCCCTTGCACCTGTAC |
| S1300312.MTP-CP11 | ACAGCTTGGGGATTCCCTG |

TABLE 2
Initial PCR and RT-PCR Method

| | | | | |
|---|---|---|---|---|
| Transcription | cDNA synthesis | 50 C. | 30 min | 1 cycle |
| | denaturation/melting | 94 C. | 2 min | |
| PCR | denaturation/melting | 94 C. | 15 sec | 30 cycles |
| | primer annealing | 48 C. | 30 sec | |
| | primer extension | 68 C. | 2 min | |
| | final extension | 68 C. | 7 min | |
| | storage | 4 C. | infinite | |

Sequencing of the mRNA was conducted by reverse transcriptase-PCR followed by Sanger sequencing. Reverse transcription of the mRNA template into cDNA and subsequent sequencing was accomplished using primers CP3, CP4, CP7 & CP9 plus multiple amplicons (Table 1). The RT-PCR program method is found in Table 3. Table 3 Primer Sequences Used for Plasmid, PCR Product, and mRNA Sequencing

TABLE 3
RT-PCR Method

| | | | | |
|---|---|---|---|---|
| Reverse | cDNA synthesis | 50 C. | 30 min | 1 cycle |
| Transcription | denaturation/melting | 94 C. | 2 min | |
| PCR | denaturation/melting | 94 C. | 15 sec | 30 cycles |
| | primer annealing | 55 C. | 45 sec | |
| | primer extension | 72 C. | 1 min | |
| | final extension | 72 C. | 10 min | |
| | storage | 4 C. | infinite | |

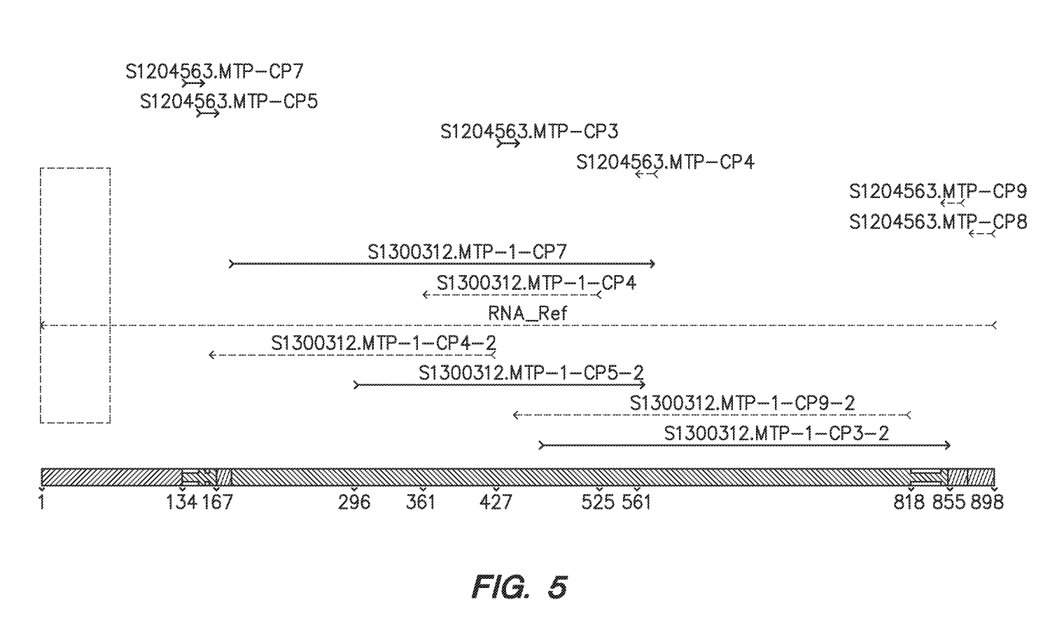

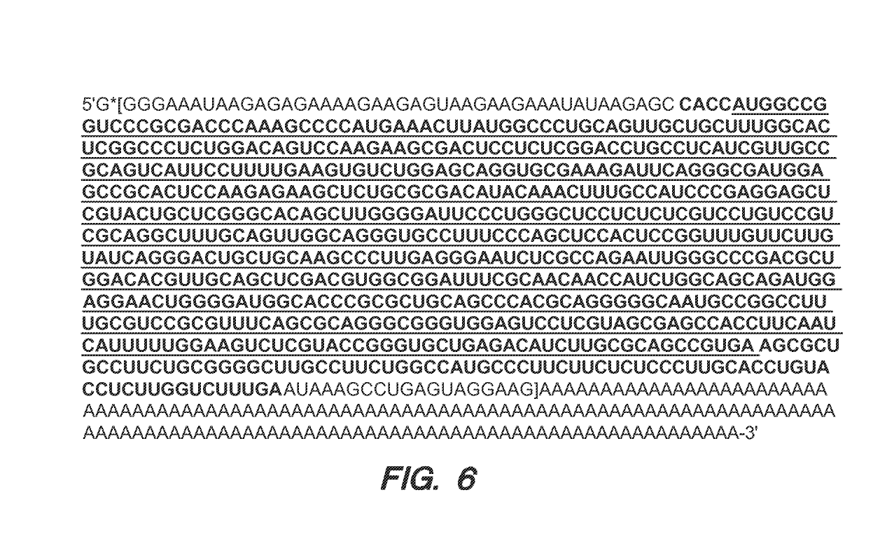

By aligning the redundantly sequenced regions of the various amplicons (FIG. 5), 100% sequence identity for 80% of the total mRNA sequence coverage (excluding the polyA tail) was observed, and 91% of the actual region (FIG. 6). There was 100% identity with 100% coverage of the protein-coding region of the mRNA (the open reading frame).

Thus, the final mRNA sequencing of a large mRNA involved amplifying the mRNA template by RT-PCR. Successful cDNA generation and sequencing was achieved with primers CP3, CP4, CP5 & CP9 (Table 1). Bidirectional sequencing results were achieved for each PCR amplified sample template using fluorescent dye-terminator chemistry and ABI Prism™ 3730xl DNA sequencers, which typically give >650 bp Q20/Phred20 read lengths.

### Example 6

### Charge Distribution Analysis

Charge heterogeneity of macromolecules is typically an indicator of structural modifications (e.g., glycosylation or deamidation of proteins; aggregation or smaller impurities in oligonucleotides. Charge heterogeneity can be assessed by ion exchange HPLC or isoelectric focusing, and charge/size heterogeneity can be assessed by capillary electrophoresis.

We developed an anion exchange HPLC to determine charge heterogeneity of mRNA because it is highly negatively charged. Since each nucleotide adds both ˜330 Da size and at least one negative charge, a later elution on AEX is indicative of also a larger size, though the resolution by size falls of dramatically as the oligonucleotide size increases beyond ˜100 nt. The analytical method is described in Table 4, and the running conditions are illustrated in Table 5.

TABLE 4
AEX Method Summary

| | |
|---|---|
| Column | 4x Dionex PAC PA-200 (250 mm), Dionex |
| | #063000 |
| Column Heater | 75° C. |
| Mobile Phase A | 25 mM Tris, 1 mM EDTA, 10% Acetonitrile, |
| | pH 8.0 |
| Mobile Phase B | 25 mM Tris, 1 mM EDTA, 800 mM NaClO₄, |
| | 10% Acetonitrile, pH 8.0 |
| Flow Rate | 1.0 mL/min |
| Injection Volume | 50 μL |
| Detection Wavelength | 260 nm |
| Total Run Time | 15 min |

TABLE 5
AEX Running Conditions

| Time (min) | Flow (mL/min) | % A | % B |
|---|---|---|---|
| Initial | 1.00 | 88 | 12 |
| 1.0 | 1.00 | 88 | 12 |
| 9.0 | 1.00 | 65 | 35 |
| 9.5 | 1.00 | 0 | 100 |
| 10.5 | 1.00 | 0 | 100 |
| 11.0 | 1.00 | 88 | 12 |
| 15.0 | 1.00 | 88 | 12 |

The use of EDTA was to chelate any divalent cations that result in potential fragmentation or self-association that would then smear chromatographic profiles. The use of high temperature is to render a more homogeneous unfolded structure as folded structural isoforms would yield a broad peak. An example AEX profile is shown in FIG. 7. Two relatively symmetrical peaks are observed even under denaturing conditions.

An orthogonal method for charge/size analysis is capillary gel electrophoresis. There are multiple modes of capillary electrophoresis separation, capillary IEF and capillary gel electrophoresis (CGE) the most commonly used for analysis of macromolecules. We have developed capillary gel electrophoresis for separation of large mRNA variants, for example poly tail containing versus tail-less. We evaluated kits from two suppliers and multiple sample preparation procedures. A critical parameter to achieve good separation and symmetrical peak shapes was complete sample denaturation for mRNA with >1000 nt length. An example of sample preparation procedure was to place mRNA in a solution containing ˜6 M urea plus 20% formamide, and then incubate for 15 min at 90° C. After such extensive mRNA denaturation, a snap cool step on ice was conducted, rather than slow cooling in the refrigerator, to minimize refolding into various structural isoforms. The Beckman Coulter dsDNA1000 kit was used. The Beckman Coulter PA800 CGE program parameters are found in Table 6.

TABLE 6
Beckman Coulter PA800 CGE Program Parameters

| Time | Parameter | Specification | Duration | | Description |
|---|---|---|---|---|---|
| | Wait | | 3 | sec | Dip capillary ends into water (wash) |
| | Rinse | 90 psi | 3 | min | Fill capillary with gel buffer |
| | | forward | | | |
| | Wait | | 3 | sec | Dip capillary ends into water (wash) |
| | Separate | 15 kV | 5 | min | Equilibrate run buffers with capillary (20 psi |
| | | | | | pressure on both sides) |
| | Wait | | 3 | sec | Dip capillary ends into water (wash) |
| | Inject | 8 kV | 5 | sec | Injection of sample |
| 0.00 | Separate | 15 kV | 50 | min | Analysis (20 psi pressure on both sides) |
| 50.00 | End | | | | End of analysis |

The gel buffers of the dsDNA1000 kit was dissolved in 20 mL of a 7 M urea solution to maintain denaturing conditions on the capillary at 50° C. Harsher conditions on the capillary by adding additional denaturing agents like formamide to the gel buffer led to fast capillary degeneration.

All experiments were performed on the Beckman Coulter PA800 CGE instrument, equipped with a temperature-controlled sample storage compartment (set to 15° C.) and a fixed wavelength UV detector. The detector wavelength was set to 254 nm (filter wheel) and injection was done electrokinetically. The CGE system operated under constant voltage conditions at 15 kV reverse mode with the capillary temperature set to 50° C.

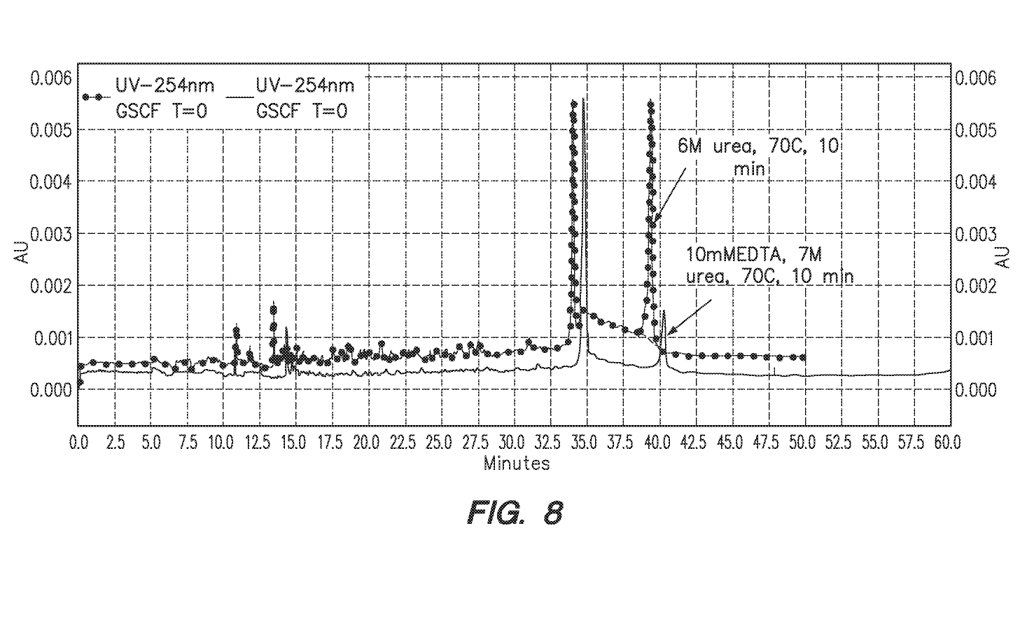

FIG. 8 shows the electropherograms of Factor IX (FIX) mRNA lacking polyA tail under denaturing conditions (6M urea, 70 C, 10 min) prior to injection and under stronger denaturing conditions. The later eluting peak converts to the earlier eluting peak under stronger denaturing conditions, indicating that there is residual structure of mRNA even in 6M urea, leading to heterogeneity in analytical methods. The tail-less FIX migrates as two species when only heated in 6M urea (top arrow) plus degradation species migrating as a broad hump in between the two peaks. Stronger denaturation unfolds the structural isoform to an earlier eluting (smaller) peak, suggestive of the large dependence of migration time and mRNA conformation. Moreover, the presence of EDTA protects from degradation during the sample handling and testing conditions.

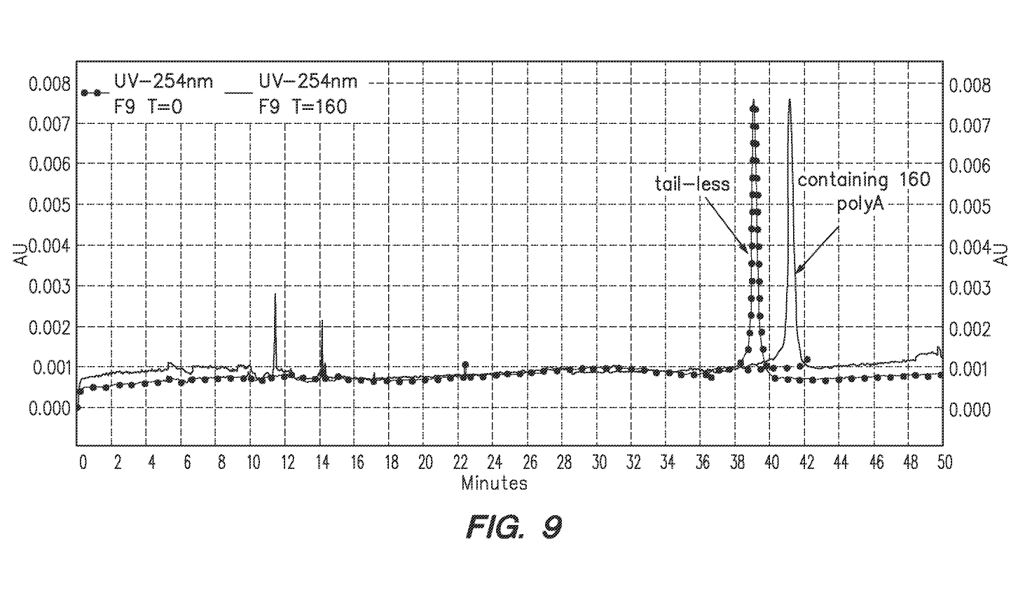

FIG. 9 shows that under the above pre-denaturation condition, the tail-less and the 160 polyA-containing FIX mRNA can be resolved. Full denaturation of samples using 70° Celsius temperatures for 10 minutes in the presence of 7M urea gave sharp peaks and migration times that separated the tail-less mRNA from the 160-polyA tail-containing mRNA.

Additional studies indicated that desalting the sample is critical for obtaining better-resolved electropherograms. Moreover, given the sample-to-sample variation in the migration time, the inclusion of RNA standard was evaluated for definitive assignment of peaks.

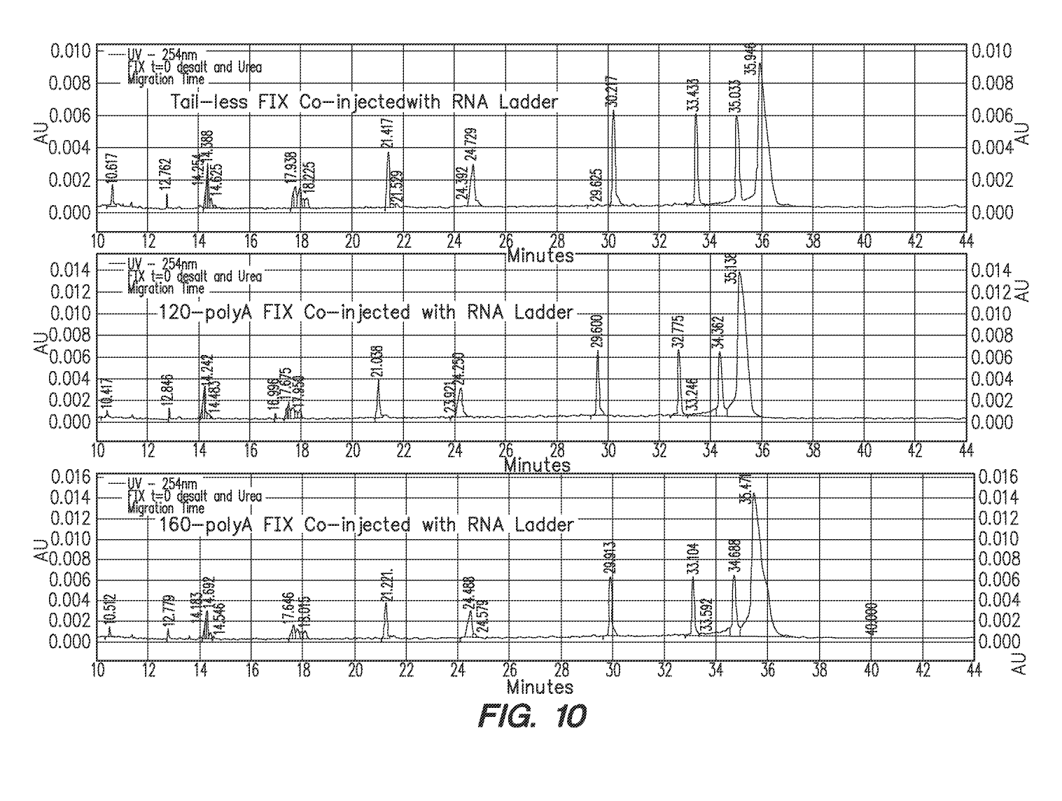

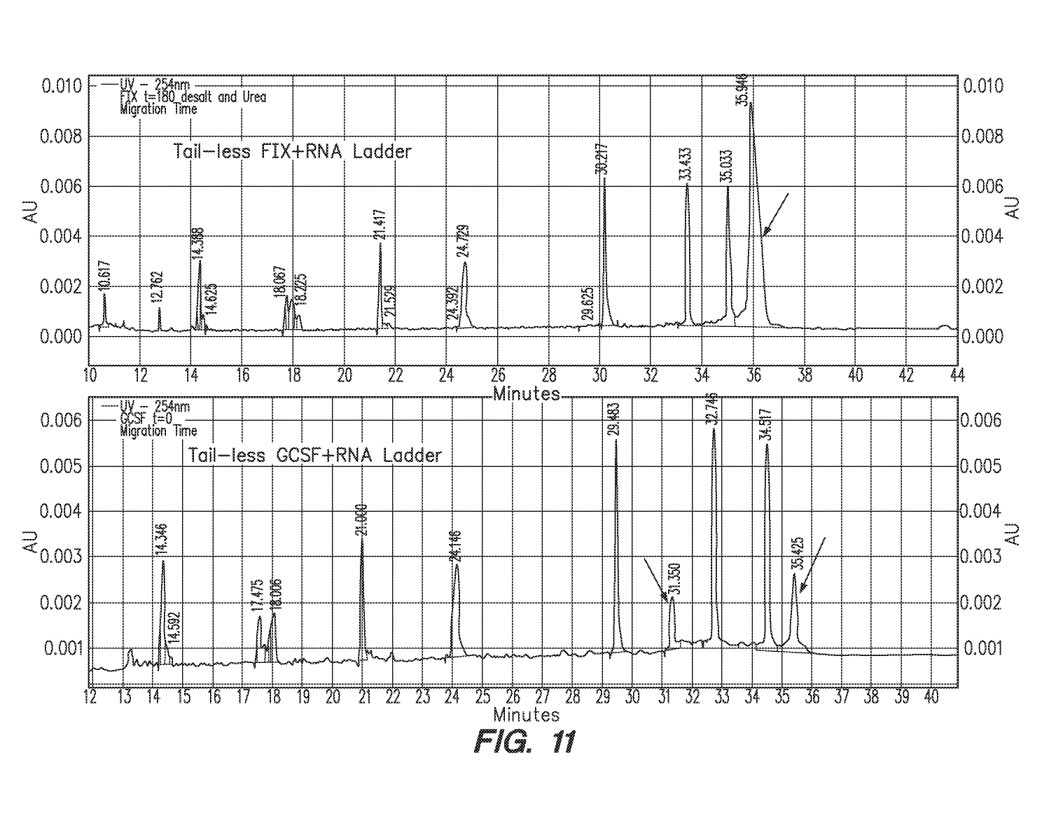

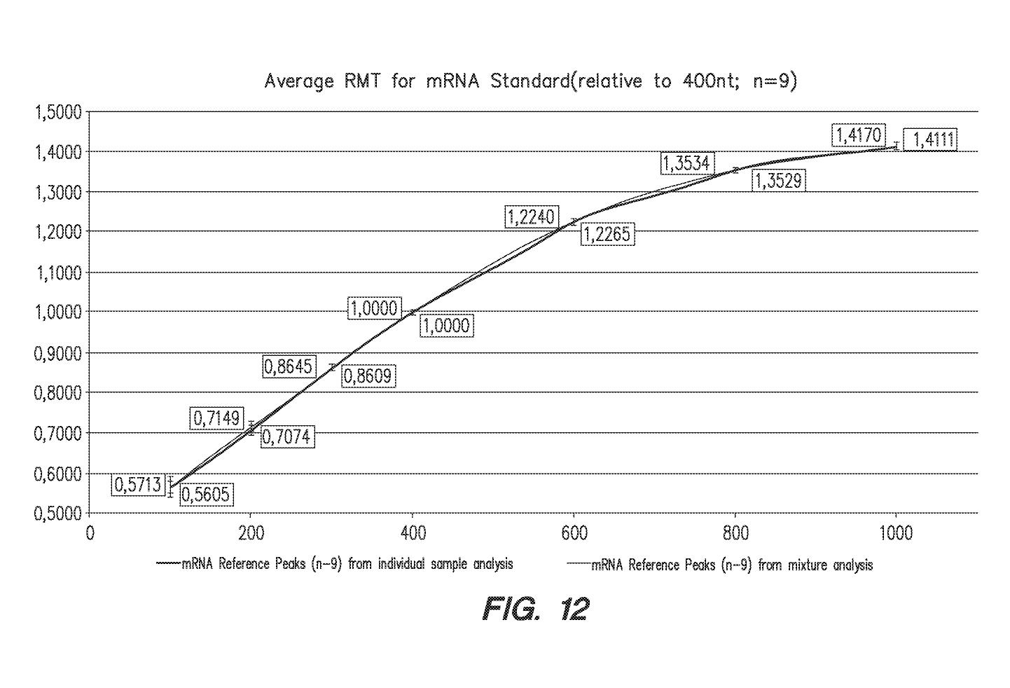

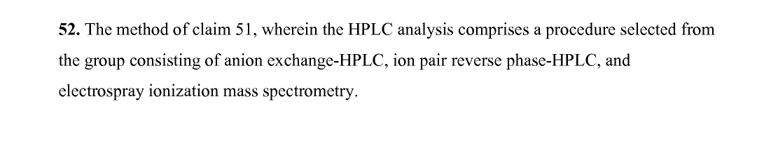

FIG. 10 shows FIX samples that were injected followed by an RNA standard. RNA standard is a mixture composed of 7 ssRNA strands with 100, 200, 300, 400, 600, 800 and 1000 bases; these were treated identical to the mRNA samples prior to spiking Injection of the mRNA standard was done at 8 kV for 1-2 sec after the electrokinetic injection of the mRNA samples at 10 kV for 8-12 sec from a separate vial. Stacking of the analytes was achieved by pressure injection (1 psi, 10 sec.) of a water plug preceding the electrokinetic sample injection step. To overcome within-run variation in the migration time of mRNA, standards were co-injected that would allow reporting of the relative migration times.

### Example 7

### Detection of RNA Impurities

Shorter mRNA sequences can be generated during in vitro transcription (IVT) of mRNA using T7 polymerase. To identify and quantify such potential product variants, they have to be separated from the intact mRNA for subsequent HPLC analysis. Since the impurities can be partially complementary to the mRNA sequence, the separation has to be done under denaturing conditions. Additionally, deoxynucleotides present in the IVT reaction or released by subsequent DNase treatment can hybridize to the mRNA, requiring their release from the mRNA by thermal or chemical denaturation. We used 7M urea to dissociate the hydrogen bonds between RNA-RNA or RNA-DNA hybrids, and followed it with spin filtration through a 50 kDa MW cut off filter that retained the mRNA and passed the smaller DNA and RNA species. Subsequently the filtrate was analysed by HPLC, either with AEX-HPLC or with IP-RP-HPLC combined with ESI-MS.

An example of the use of this procedure is demonstrated with GCSF mRNA, a single stranded mRNA containing 899 nucleotides. As a marker of the efficiency of denaturation/filtration procedure, a 40mer single stranded DNA complementary to positions 860-899 of the GCSF mRNA coding sequence and a 40mer single stranded RNA impurity marker complementary to positions 2-41 of the GCSF mRNA coding sequence were synthesized.

| Sample | | Comple- |
|---|---|---|
| ID | Sequence (5′->3′) | mentarity |
| X01310K1 | CTTCCTACTCAGGCTTTATTCAAAGAC | 860 to 899 |
| | CAAGAGGTACAGG | |
| X01311K1 | CUUAUAUUUCUUCUUACUCUUCUUUUC | 2 to 41 |
| | UCUCUUAUUUCCC | |

Solutions of mRNA were prepared in 1×TE buffer at a concentration of at least 0.7 mg/mL (˜2.5 μM). DNA and RNA impurity marker stock solutions were prepared at a concentration of 25 μM. The solutions can be stored at −20° C. A mixture of 25 μL of mRNA stock and 175 μL of 8 M urea solution containing 10 mM EDTA and 50 mM TEA acetate was prepared (final 7M urea). Alternatively, for stronger denaturation and higher impurity concentration, 10 μL of a 5 mg/mL mRNA solution can be diluted with 190 μL of the 8M urea solution, resulting in a final concentration of −0.25 mg/mL mRNA in 7.6 M urea.

Next, 200 μL of the above solution was heated to 90° C. for 10 minutes in a screw cap vial and subsequently snap cooled on ice. The Sartorius Vivaspin 500 spin filter devices were washed three times before use. For each wash step 500 μL of the 8 M urea solution containing 10 mM EDTA and 50 mM TEAAc was placed into the spin filter devices and centrifuged for 10 minutes at 1000×g. The filtrated of the first two washes were discarded, and the third filtrate was used as a matrix blank.

After the third wash the residual solution in the spin filter device was decanted and then 200 μL of the snap cooled mRNA solution was placed into the filter device. The solution was centrifuged for 3-5 minutes at 1000×g.

mRNA Nucleotide Variant Analysis (Outcome of Hybridrization):

mRNA preparations might contain trace amounts of aberrant nucleotides, for example deaminated, depurinated, or oxidized nucleotides. Identifying the low level single modifications in a large mRNA requires specific and sensitive methods. RT-Sanger is generally not sensitive enough for detection of such species. To characterize potential aberrant or degraded nucleotides, the mRNA is treated with nuclease P1 or other 3′-exonucleases such as snake venom phosphodiesterase, and the released nucleotides is characterized. Requirement for the initiation of the digestion reaction is a free 3′-OH at the first 3′-nucleotide of the mRNA (no 3′- or 2′-3′-cyclic phosphate). Digestion is combined with bovine alkaline phosphatase (BAP) treatment to generate the nucleosides from the released nucleotides. Nucleosides with unexpected masses are then further characterized by MS/MS analysis to define the structure. Once the structure is identified, standards are made of these nucleotides and used for quantifying trace levels in mRNA preparations.

Analysis of Purity with Respect to Duplexes:

Using RNases that target duplex structures in mRNA is applied to determining the purity of the mRNA with respect to duplex structures. For example, Figure X shows that in addition to release of polyA tail, some earlier eluting species are also observed which might be indicative of the presence of other duplexes in the mRNA.

Site-Specific Cleavage by Other Enzymes:

A number of other enzymes that target specific sequences or duplex sites can be investigated to obtain a comprehensive analysis of the primary sequence. Examples are RNase T1, U, S, and MazF.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES CITED

Oberacher H, Pitterl F. On the use of ESI-QqTOF-MS/MS for the comparative sequencing of nucleic acids. Biopolymers. 2009 June; 91(6):401-9.
Yi-Fen J. Chrom 1990 508:61-73.
Apffel and Hancock Nucleic Acids Res. 2009 November; 37(21).
Thomas P. Shields, Emilia Mollova, Linda Ste. Marie, Mark R. Hansen, and Arthur Pardi, High-performance liquid chromatography purification of homogenous-length RNA produced by trans cleavage with a hammerhead ribozyme RNA (1999), 5:1259-1267
Amy C. Anderson, Stephen A. Scaringe, Brandon E. Earp, and Christin A. Frederick, HPLC Purification of RNA for Crystallography and NMR of RNA (1996), 2:110-117.
Masato Taoka, Yoshio Yamauchi, Yuko Nobe, Shunpei Masaki, Hiroshi Nakayama, Hideaki Ishikawa, Nobuhiro Takahashi, and Toshiaki Isobe. An analytical platform for mass spectrometry-based identification and chemical analysis of RNA in ribonucleoprotein complexes. Nucleic Acids Res. 2009 November; 37(21)
Matthieson 2009, use of software to identify RNA with specific fragmentation pattern by RNase (e.g. T1)
Lapham J, Crothers D M, 1996. RNase H cleavage for processing of in vitro transcribed RNA for NMR studies and RNA ligation. RNA 2:289-296
Lapham J, Yu Y T, Shu M D, Steitz J A, Crothers D M. 1997. The position of site-directed cleavage of RNA using RNase H and 29-Omethyloligonucleotides is dependent on the enzyme source. RNA 3:950-951

1. A method for characterizing an RNA transcript, comprising:
obtaining the RNA transcript; and
characterizing the RNA transcript using a procedure selected from the group consisting of oligonucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA transcript.

2. The method of claim 1, wherein the RNA transcript is the product of in vitro transcription using a non-amplified DNA template.

3. The method of claim 1, wherein the procedure is oligonucleotide mapping comprising:
contacting the RNA transcript with a plurality of nucleotide probes under conditions sufficient to allow hybridization of the nucleotide probes to the RNA transcript to form duplexes, wherein each of the nucleotide probes comprises a sequence complementary to a different region of the RNA transcript;
contacting the duplexes with an RNase under conditions sufficient to allow RNase digestions of the duplexes to form reaction products;
analyzing the reaction products using a procedure selected from the group consisting of reverse phase high performance liquid chromatography (RPHPLC), anion exchange HPLC (AEX), and RP-HPLC coupled to mass spectrometry (MS); and
using the analysis of the reaction products to determine the sequence of the RNA transcript, thereby characterizing the RNA transcript.

4. The method of claim 3, wherein the RNase is RNase H or RNase T1.

5. The method of claim 3, wherein the nucleotide probes are between 10 and 40 nucleotides in length.

6. The method of claim 3, wherein the nucleotide probes are between 15 and 30 nucleotides in length.

7. The method of claim 5 or 6, wherein the nucleotide probes comprise at least 8 deoxynucleotides.

8. The method of claim 3, wherein at least one of the nucleotide probes comprises a region that is complementary to a region adjacent to the poly-A tail of the RNA transcript.

9. The method of claim 3, wherein the nucleotide probes are complementary to regions no more than 50 nucleotides apart along the RNA transcript.

10. The method of claim 3, wherein the RNA transcript is a full length RNA transcript.

11. The method claim 3, wherein the RNA transcript comprises chemically modified ribonucleotides.

12. The method of claim 3, wherein the RNA transcript is between 100 and 10,000 nucleotides in length.

13. The method of claim 3, wherein the RNA transcript is between 600 and 10,000 nucleotides in length.

14. The method of claim 3, wherein the RNA transcript is between 700 and 3,000 nucleotides in length.

15. The method of claim 1, wherein the procedure is reverse transcriptase sequencing comprising:
contacting the RNA transcript with a reverse transcriptase, a set of primers, and deoxyribonucleotides to obtain one or more cDNA samples;
contacting the one or more cDNA samples with a second set of primers under conditions sufficient to allow peR to occur, wherein the cDNA sample is a template for obtaining a product comprising amplified cDNA;
analyzing the product using a DNA sequencing procedure; and
using the analysis of the product to determine the sequence of the RNA transcript, thereby characterizing the RNA transcript.

16. The method of claim 15, wherein the DNA sequencing procedure comprises Sanger sequencing.

17. The method of claim 15, wherein the DNA sequencing procedure comprises bidirectional sequencing.

18. The method of claim 15, wherein the primers are complementary to the untranslated regions of mRNA.

19. The method of claim 15, wherein the primers are selected from the group having sequences comprising: CGTCGAGCTGCAACGTG, CGTCCTGTCCGTCGCAG, TTTTTTTCTTCCTACTCAGGC, and GAAATATAAGAGCCACCATGG.

20. The method of claim 15, wherein the RNA transcript is a full length RNA transcript.

21. The method of claim 15, wherein the RNA transcript comprises chemically modified ribonucleotides.

22. The method of claim 15, wherein the RNA transcript is between 100 and 10,000 nucleotides in length.

23. The method of claim 15, wherein the RNA transcript is between 600 and 10,000 nucleotides in length.

24. The method of claim 15, wherein the RNA transcript is between 700 and 3,000 nucleotides in length.

25. The method of claim 1, wherein the procedure is charge distribution analysis comprising a second procedure selected from the group consisting of: anion exchange HPLC (AEX) and capillary electrophoresis.

26. The method of claim 25, wherein the capillary electrophoresis is capillary gel electrophoresis.

27. The method of claim 25, wherein the RNA transcript is between 100 and 10,000 nucleotides in length.

28. The method of claim 25, wherein the RNA transcript is between 600 and 10,000 nucleotides in length.

29. The method of claim 25, wherein the RNA transcript is between 700 and 3,000 nucleotides in length.

30. The method of claim 25, wherein the RNA transcript is a full length RNA transcript.

31. The method of claim 25, wherein the RNA transcript comprises chemically modified ribonucleotides.

32. The method of claim 25, wherein the second procedure is AEX comprising:
contacting a sample comprising the RNA transcript with an ion exchange sorbent comprising a positively-charged functional group linked to solid phase media, the sample delivered with at least one mobile phase, wherein the RNA transcript in the sample binds the positively-charged functional group of the ion exchange sorbent;
eluting from the ion exchange sorbent a portion of the sample comprising the RNA transcript and one or more separate portions of the sample comprising any impurities;
analyzing at least one aspect of the portion of the sample comprising the RNA transcript and the one or more separate portions of the sample comprising the impurities, wherein the at least one aspect is selected from the group consisting of charge heterogeneity of the RNA transcript, mass heterogeneity of the RNA transcript, process intermediates, impurities, and degradation products; and
using the analysis of the at least one aspect of the portion of the sample comprising the RNA transcript and the one or more separate portions of the sample comprising the impurities to determine the charge heterogeneity of the RNA transcript, thereby characterizing the RNA transcript.

33. The method of claim 32, wherein the sample is delivered under denaturing conditions.

34. The method of claim 33, wherein the denaturing conditions comprise contacting the sample with urea.

35. The method of claim 32, wherein the at least one mobile phase is a TrisEDTA-acetonitrile buffered mobile phase.

36. The method of claim 32, wherein the at least one mobile phase comprises two Tris-EDTA-acetonitrile buffered mobile phases.

37. The method of claim 32, wherein the at least one mobile phase comprises a chaotropic salt.

38. The method of claim 37, wherein the chaotropic salt is sodium perchlorate.

39. The method of claim 25, wherein the second procedure is capillary gel electrophoresis comprising:
delivering a sample comprising the RNA transcript into a capillary with an electrolyte medium;
applying an electric field to the capillary that causes the RNA transcript to migrate through the capillary, wherein the RNA transcript has a different electrophoretic mobility than any impurities such that the RNA transcript migrates through the capillary at a rate that is different from a rate at which the impurities migrate through the capillary;
collecting from the capillary a portion of the sample comprising the RNA transcript and one or more separate portions of the sample comprising the impurities;
analyzing at least one aspect of the portion of the sample comprising the RNA transcript and the one or more separate portions of the sample comprising the impurities, wherein the at least one aspect comprises charge heterogeneity of the RNA transcript; and
using the analysis of the at least one aspect of the portions of the sample comprising the RNA transcript and the one or more separate portions of the sample comprising the impurities to determine the charge distribution of the RNA transcript and the impurities, thereby characterizing the RNA transcript.

40. The method of claim 39, wherein the electrophoretic mobility of the RNA transcript is proportional to a mass and an ionic charge of the RNA transcript and inversely proportional to frictional forces in the electrolyte medium.

41. The method of claim 39, wherein the sample is delivered under denaturing conditions.

42. The method of claim 1, wherein the procedure is detection of RNA impurities comprising: detecting short mRNA transcripts, detecting RNA-RNA and RNA-DNA hybrids, and detecting aberrant nucleotides.

43. The method of claim 42, wherein the RNA transcript is a full length RNA transcript.

44. The method of claim 42, wherein the RNA transcript comprises chemically modified ribonucleotides.

45. The method of claim 42, wherein the RNA transcript is between 100 and 10,000 nucleotides in length.

46. The method of claim 42, wherein the RNA transcript is between 600 and 10,000 nucleotides in length.

47. The method of claim 42, wherein the RNA transcript is between 700 and 3,000 nucleotides in length.

48. The method of claim 42, wherein detecting short mRNA transcripts comprises:
denaturing the RNA transcript; and
subjecting the denatured RNA transcript to HPLC analysis, whereby the HPLC analysis quantifies any short mRNA transcript impurities.

49. The method of claim 48, wherein the HPLC analysis comprises reverse phase HPLC.

50. The method of claim 49, wherein the reverse phase HPLC analysis is followed by tandem mass spectrometry, whereby the tandem mass spectrometry identifies any impurities.

51. The method of claim 42, wherein detecting RNA-RNA and RNA-DNA hybrids comprises:
subjecting the RNA transcript to treatment with urea and EDTA;
subjecting the treated RNA transcript to spin filtration, wherein the filtrate retains a product comprising the impurities;
analyzing the product using HPLC; and
using the analysis of the product to determine the purity of the RNA transcript, whereby the analysis comprises identification of any RNA-RNA and RNA-DNA hybrids in the product, thereby characterizing the RNA transcript.

52. The method of claim 51, wherein the HPLC analysis comprises a procedure selected from the group consisting of anion exchange-HPLC, ion pair reverse phase-HPLC, and electrospray ionization mass spectrometry.


SEQUENCE LISTING (21 sequences)

SEQ ID NO: 1 - DNA, 17 bp, Artificial Sequence
  Synthetic primer
cgtcgagctgcaacgtg

SEQ ID NO: 2 - DNA, 17 bp, Artificial Sequence
  Synthetic primer
cgtcctgtccgtcgcag

SEQ ID NO: 3 - DNA, 21 bp, Artificial Sequence
  Synthetic primer
tttttttcttcctactcaggc

SEQ ID NO: 4 - DNA, 21 bp, Artificial Sequence
  Synthetic primer
gaaatataagagccaccatgg

SEQ ID NO: 5 - DNA, 300 bp, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - DNA, 120 bp, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 120 bp, Artificial Sequence
  Synthetic polynucleotide
tttttttttttttttttttttttttttttttttttttttttttttttttttttttttttttttttttttttttttttttttttttttttttttttttttttttttttttttttttttttt

SEQ ID NO: 8 - DNA, 18 bp, Artificial Sequence
  Synthetic oligonucleotide
uuuuuttttcttccuacu

SEQ ID NO: 9 - DNA, 18 bp, Artificial Sequence
  Synthetic oligonucleotide
uucuucctactcaggcuu

SEQ ID NO: 10 - DNA, 21 bp, Artificial Sequence
  Synthetic primer
cattcaaatatgtatccgctc

SEQ ID NO: 11 - DNA, 18 bp, Artificial Sequence
  Synthetic primer
gagaaaaaagcaacgcac

SEQ ID NO: 12 - DNA, 18 bp, Artificial Sequence
  Synthetic primer
caggctttattcaaagac

SEQ ID NO: 13 - DNA, 17 bp, Artificial Sequence
  Synthetic primer
ggaccctcgtacagaag

SEQ ID NO: 14 - DNA, 23 bp, Artificial Sequence
  Synthetic primer
ggaaataagagagaaaagaagag

SEQ ID NO: 15 - DNA, 19 bp, Artificial Sequence
  Synthetic primer
ctctcccttgcacctgtac

SEQ ID NO: 16 - DNA, 19 bp, Artificial Sequence
  Synthetic primer
acagcttggggattccctg

SEQ ID NO: 17 - DNA, 40 bp, Artificial Sequence
  Synthetic oligonucleotide
cttcctactcaggctttattcaaagaccaagaggtacagg

SEQ ID NO: 18 - RNA, 40 nt, Artificial Sequence
  Synthetic oligonucleotide
cuuauauuucuucuuacucuucuuuucucucuuauuuccc

SEQ ID NO: 19 - DNA, 1430 bp, Artificial Sequence
  Synthetic polynucleotide
ataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttttgacgcgacttcgaatagggcgaattgggccctctagatgcatgctcgagcggccgccttcctactcaggctttattcaaagaccaagaggtacaggtgcaagggagagaagaagggcatggccagaaggcaagccccgcagaaggcagcgcttcacggctgcgcaagatgtctcagcacccggtacgagacttccaaaaatgattgaaggtggctcgctacgaggactccacccgccctgcgctgaaacgcggacgcaaaggccggcattgccccctgcgtgggctgcagcgcgggtgccatccccagttcctccatctgctgccagatggttgttgcgaaatccgccacgtcgagctgcaacgtgtccagcgtcgggcccaattctggcgagattccctcaagggcttgcagcagtccctgatacaagaacaaaccggagtggagctgggaaaggcaccctgccaactgcaaagcctgcgacggacaggacgagagaggagcccagggaatccccaagctgtgcccgagcagtacgagctcctcgggatggcaaagtttgtatgtcgcgcagagcttctcttggagtgcggctccatcgccctgaatctttcgcacctgctccagacacttcaaaaggaatgactgcggcaacgatgaggcaggtccgagaggagtcgcttcttggactgtccagagggccgagtgccaaagcagcaactgcagggccataagtttcatggggctttgggtcgcgggaccggccatggtggctcttatatttcttcttactcttcttttctctcttatttccctatagtgagtcgtattagcttctgtacgagggtccaaaagctttcagcgaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggtt

SEQ ID NO: 20 - DNA, 787 bp, Artificial Sequence
  Synthetic polynucleotide
cttcctactcaggctttattcaaagaccaagaggtacaggtgcaagggagagaagaagggcatggccagaaggcaagccccgcagaaggcagcgcttcacggctgcgcaagatgtctcagcacccggtacgagacttccaaaaatgattgaaggtggctcgctacgaggactccacccgccctgcgctgaaacgcggacgcaaaggccggcattgccccctgcgtgggctgcagcgcgggtgccatccccagttcctccatctgctgccagatggttgttgcgaaatccgccacgtcgagctgcaacgtgtccagcgtcgggcccaattctggcgagattccctcaagggcttgcagcagtccctgatacaagaacaaaccggagtggagctgggaaaggcaccctgccaactgcaaagcctgcgacggacaggacgagagaggagcccagggaatccccaagctgtgcccgagcagtacgagctcctcgggatggcaaagtttgtatgtcgcgcagagcttctcttggagtgcggctccatcgccctgaatctttcgcacctgctccagacacttcaaaaggaatgactgcggcaacgatgaggcaggtccgagaggagtcgcttcttggactgtccagagggccgagtgccaaagcagcaactgcagggccataagtttcatggggctttgggtcgcgggaccggccatggtggctcttatatttcttcttactcttcttttctctcttatttccctatagtgagtcgtattagcttctgtacga

SEQ ID NO: 21 - RNA, 899 nt, Artificial Sequence
  Synthetic polynucleotide
ggggaaauaagagagaaaagaagaguaagaagaaauauaagagccaccauggccggucccgcgacccaaagccccaugaaacuuauggcccugcaguugcugcuuuggcacucggcccucuggacaguccaagaagcgacuccucucggaccugccucaucguugccgcagucauuccuuuugaagugucuggagcaggugcgaaagauucagggcgauggagccgcacuccaagagaagcucugcgcgacauacaaacuuugccaucccgaggagcucguacugcucgggcacagcuuggggauucccugggcuccucucucguccuguccgucgcaggcuuugcaguuggcagggugccuuucccagcuccacuccgguuuguucuuguaucagggacugcugcaagcccuugagggaaucucgccagaauugggcccgacgcuggacacguugcagcucgacguggcggauuucgcaacaaccaucuggcagcagauggaggaacuggggauggcacccgcgcugcagcccacgcagggggcaaugccggccuuugcguccgcguuucagcgcagggcggguggaguccucguagcgagccaccuucaaucauuuuuggaagucucguaccgggugcugagacaucuugcgcagccgugaagcgcugccuucugcggggcuugccuucuggccaugcccuucuucucucccuugcaccuguaccucuuggucuuugaauaaagccugaguaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa